(19) United States
(12) Patent Application Publication
(10) Pub. No.: US 2026/0048150 A1
(43) Pub. Date: Feb. 19, 2026

(54) METHODS AND SYSTEMS FOR IMPROVED NUCLEIC ACID DELIVERY VIA ULTRASOUND

(71) Applicant: SonoThera, Inc., South San Francisco, CA (US)

(72) Inventors: FEINSTEIN B. Steven, Highland Park, IL (US); GREENBERG Kenneth, Berkeley, CA (US); CAMPBELL Barry, San Mateo, CA (US); KRIVEGA Ivan, South San Francsico, CA (US)

(21) Appl. No.: 19/368,749

(22) Filed: Oct. 24, 2025

Related U.S. Application Data
(63) Continuation of application No. PCT/US2024/026239, filed on Apr. 25, 2024.
(60) Provisional application No. 63/462,508, filed on Apr. 27, 2023.
(60) Provisional application No. 63/625,279, filed on Jan. 25, 2024.

(51) Int. Cl. A61K 48/00; A61K 31/713

(52) U.S. Cl. CPC A61K 48/0083; A61K 31/713; A61K 48/0008

(57) ABSTRACT

An effective gene therapy technique that can transfect a gene to a cell in an organ or a tissue in a subject in a safe, effective, and durable manner is disclosed herein. The methods disclosed herein can induce expression of a nucleic acid payload in target cell(s) of a mammalian subject by administering to the subject a linear double stranded DNA (dsDNA) construct covalently closed at each of its ends by DNA loops, the linear dsDNA construct comprising the nucleic acid payload; administering to the subject a plurality of microbubbles; and administering to the subject, in proximity to the target cell(s) an effective amount of an ultrasound energy, thereby inducing the formation of pores the target cell(s) by disrupting the plurality of microbubbles to result in introduction of the dsDNA into the target cell(s) and expression of the nucleic acid payload in the target cell(s).

### CROSS REFERENCE TO RELATED APPLICATIONS

This application is a continuation of PCT Patent Application No. PCT/US2024/026239 which claims the benefit of U.S. Provisional Ser. No. 63/462,508 filed Apr. 27, 2023, and U.S. Provisional Ser. No. 63/625,279 filed Jan. 25, 2024, each of which is incorporated herein by reference in its entirety and for all purposes.

### BACKGROUND

Gene therapy, in which a functional copy of a gene is transfected into a cell, has been proposed as a possible method of treating genetic diseases. However, prior art methods of gene therapy using ultrasound or sonoporation suffer from significant shortcomings such as substantial cellular damage, inflammation, and/or death resulting from ultrasound treatment, or immune responses due to viral vector-based gene therapies, which have prevented the clinical development and commercialization of these methodologies. There remains a need in the art for an effective gene therapy technique that can transfect a gene to a cell in an organ or a tissue in a subject in a safe, effective, and durable manner.

### SUMMARY

One challenge appreciated by the inventors in sonoporation based gene therapy methods includes maintaining durable expression of the transgene in the weeks following transfection. For example, the use of conventional nucleotide constructs (e.g., plasmid DNA constructs) in sonoporation-mediated gene therapy can result in low transfection rates, undetectable levels of therapeutic protein expression in the days or weeks following a sonoporation treatment, or in short-duration of protein expression which is initially detectable but which lacks durability of expression. It is appreciated by the inventors there remains a need in the art for improved processes which permit sonoporation gene therapy treatments that can improve gene transfection and gene expression of a nucleic acid payload in a cell over an extended period following a single sonoporation treatment. As such, disclosed herein are improved methods for sonoporation based gene therapy treatments which utilize next-generation DNA constructs, which increase gene transfection and expression, and which increase the durability of gene expression following sonoporation based gene therapy treatments. The methods disclosed herein can include administering a linear double stranded DNA (dsDNA) construct covalently closed at each of its ends by DNA loops, the linear dsDNA construct comprising the nucleic acid payload, the treatment resulting in sustained expression of a nucleotide payload. The methods of administering a linear double stranded DNA (dsDNA) construct to a subject using sonoporation can induce a steady state of gene expression for weeks following the sonoporation treatment under circumstances where expression of a payload encoded by a pDNA construct would fail to maintain sustained gene expression. The methods disclosed herein can further include administering treatment sessions at particular intervals and applying ultrasound energy using reduced power, reduced mechanical indexes, alternating between high and low mechanical indexes, in short flash pulses, and/or in short treatment sessions to achieve further beneficial results.

Aspects disclosed herein provide a method of inducing an expression of a nucleic acid payload in target cell(s) of a mammalian subject comprising: administering to the subject a linear double stranded DNA (dsDNA) construct covalently closed at each of its ends by DNA loops, the linear dsDNA construct comprising the nucleic acid payload; administering to the subject a plurality of microbubbles; and administering to the subject, in proximity to the target cell(s) an effective amount of an ultrasound energy, thereby inducing formation of pores the target cell(s) by disrupting the plurality of microbubbles to result in introduction of the dsDNA into the target cell(s) and expression of the nucleic acid payload in the target cell(s). In some embodiments, the linear dsDNA construct comprising the nucleic acid payload is administered at a first dosage that is less than, by mass, a dosage of DNA construct of a plasmid (pDNA) construct comprising the nucleic acid payload required to induce an equivalent level of payload expression in the subject when administered the effective amount of the ultrasound energy. In some embodiments, the linear dsDNA construct is administered to the subject at a dosage of up to about 0.2 mg/kg bodyweight. In some embodiments, the first dosage of the dsDNA construct is at least 10%, 20%, 30% 40%, 50%, 60% or 70%, less than a second dosage of the plasmid (pDNA) construct. In some embodiments, the linear dsDNA construct is administered at a first dosage that is less than 500 micrograms. In some embodiments, the linear dsDNA construct is administered at a first dosage that is less than 1.5 mg/kg bodyweight. In some embodiments, the combination of (1) administering to the subject the dsDNA construct covalently closed at each of its ends by DNA loops; (2) administering to the subject the plurality of microbubbles; and (3) administering to the subject the effective amount of an ultrasound energy, thereby inducing expression of the nucleic acid payload in the target cell(s) induces expression of the nucleic acid payload within 24 hours. In some embodiments, the expression of a protein encoded by the nucleic acid payload in the target cell(s) is maintained for at least 1, 2, 3, 4, 5, 6, 7, 8, 9, 10, 11, 12, 13, 14, 15, 16, 17, 18, 19, 20, 21, 22, 23, 24, 25, or 26 weeks following performance of a.-c. In some embodiments, the expression of a protein encoded by the nucleic acid payload in the target cell(s) is maintained for at least 1, 2, 3, 4, 5, 6, 7, 8, 9, 10, 11, 12, 13, 14, 15, 16, 17, 18, 19, 20, 21, 22, 23, 24, 25, or 26 weeks following a single performance of a.-c. In some embodiments, the expression of a protein encoded by the nucleic acid payload in the target cell(s) is maintained at an elevated level as compared to a baseline expression level for at least 1, 2, 3, 4, 5, 6, 7, 8, 9, 10, 11, 12, 13, 14, 15, 16, 17, 18, 19, 20, 21, 22, 23, 24, 25, or 26weeks following the combination of (1) administering to the subject the dsDNA construct covalently closed at each of its ends by DNA loops; (2) administering to the subject the plurality of microbubbles; and (3) administering to the subject the effective amount of an ultrasound energy. In some embodiments, the expression of a protein encoded by the nucleic acid payload in the target cell(s) is maintained at a steady state at an elevated level as compared to a baseline expression level starting at least 3 weeks following the combination of (1) administering to the subject the dsDNA construct covalently closed at each of its ends by DNA loops; (2) administering to the subject the plurality of microbubbles; and (3) administering to the subject the effective amount of an ultrasound energy. In some embodiments, the expression of a protein encoded by the nucleic acid payload in the target cell(s) is maintained at a steady state at an elevated level as compared to a baseline expression level starting at least 3 weeks following performance of a.-c., and continuing to hold at the steady state thereafter for at least 1, 2, 3, 4, 5, 6, 7, 8, 9, 10, 11, 12, 13, 14, 15, 16, 17, 18, 19, 20, 21, 22, 23, 24, 25, or 26 weeks. In some embodiments, the steady state at the elevated level is within 1 order of magnitude of a peak level of expression. In some embodiments, the administering the effective amount of ultrasound energy comprises continuously applying ultrasound energy for a duration of a treatment session. In some embodiments, continuously applying ultrasound energy comprises applying ultrasound energy at a first mechanical index for a first duration, applying ultrasound energy at a second mechanical index for a second duration, and re-applying ultrasound energy at the first mechanical index without ceasing application of ultrasound energy for the duration of a treatment session. In some embodiments, the administering the nucleic acid payload or administering the plurality of microbubbles comprises administering intravenously through a peripheral vein. In some embodiments, the linear dsDNA construct is administered to the subject at a dosage between 0.4 mg/kg bodyweight to 1.5 mg/kg bodyweight. In some embodiments, the linear dsDNA construct is administered to the subject at a concentration of about 3.5 μg/μl. In some embodiments, the target cell(s) comprise hepatic cells. In some embodiments, the target cell(s) are in a liver. In some embodiments, the target cell(s) comprise renal cells. In some embodiments, the target cell(s) are in a is a kidney. In some embodiments, the target cell(s) comprise pancreatic cells. In some embodiments, the linear dsDNA construct comprises a protelomerase recognition sequence in each loop. In some embodiments, the protelomerase recognition sequence comprises at least 14 nucleotides of a double stranded palindromic sequence. In some embodiments, the linear dsDNA construct comprises at least one eukaryotic promoter. In some embodiments, the linear dsDNA construct comprises at least one stem loop motif comprising a central non-complementary loop section flanked by two complementary sequences. In some embodiments, the loops are hairpin loops. In some embodiments, the microbubbles comprise a protein stabilized shell. In some embodiments, the microbubbles comprise an albumin stabilized shell. In some embodiments, the microbubbles comprise perflutren gas. In some embodiments, the microbubble is an Optison microbubble. In some embodiments, the microbubbles comprise a lipid stabilized shell. In some embodiments, a concentration of the microbubbles administered is up to about 1.2*10\hat{ }10 microbubbles/ml. In some embodiments, the microbubbles are administered at a concentration of at least 5*10\hat{ }8 microbubbles/ml. In some embodiments, a concentration of the microbubbles administered is between 5*10\hat{ }8 and 8*10\hat{ }8 microbubbles/ml. In some embodiments, the administering ultrasound energy occurs transcutaneously. In some embodiments, the administering the effective amount of the ultrasound energy comprises administering at least 5 ultrasound flashes at a second mechanical index which is increased relative to a first mechanical index, the ultrasound flashes at the second mechanical index being administered less than 10 seconds apart from one another. In some embodiments, the administering the effective amount of the ultrasound energy comprises administering a plurality of ultrasound flashes at an increased mechanical index, the ultrasound flashes at the increased mechanical index being administered about 1 s to about 10 s apart from one another. In some embodiments, the administering the effective amount of the ultrasound energy comprises administering a plurality of ultrasound flashes at an increased mechanical index, with each of the plurality of ultrasound flashes at the increased mechanical index being administered about 5 s to about 10 s apart from one another. In some embodiments, the administering the effective amount of the ultrasound energy comprises administering a plurality of ultrasound flashes at an increased mechanical index, the ultrasound flashes at the increased mechanical index being administered 1 s to about 5 s apart from one another. In some embodiments, the ultrasound flash(s) is an application of ultrasound at the increased mechanical index or the second mechanical index over a time period. In some embodiments, the time period is less than 1 s. In some embodiments, the time period is from about 0.7 us to about 3 us (microseconds). In some embodiments, the time period is about 0.72, 0.82, 0.72, 0.98, or 2.28 us. In some embodiments, the administering of the ultrasound energy of c. comprises administering at least 5 ultrasound flashes at the second mechanical index. In some embodiments, the nucleic acid payload comprises a therapeutic transgene. In some embodiments, the therapeutic transgene comprises: GLP-1, INS, Reg3g, MafA, PDX-1, NUEROG3, NGN3, DRYK, DYRK1A, DYRK1B, Factor VIII, Factor IX, PKD1, PKD2, COL4A3, COL4A4, COL4A5, Klotho, Smad7, TGF-beta, SLC7A1, SLC3A9, UMOD, REN, HNF1B, MUC1, SLC12A1, KCNJ1, CLCNKA, CLCNKB, BSND, MAGED2, NPHS1, NPHS2, CTNS or combinations thereof. In some embodiments, the nucleic acid payload comprises the therapeutic transgene is coupled to a promoter sequence comprising: ApoE promoter, CAG promoter, AAT promoter, or combinations thereof. In some embodiments, the nucleic acid payload and the plurality of microbubbles are administered in a volumetric ratio of at least 1-part nucleic acid payload solution to 4 parts microbubble solution. In some embodiments, the ultrasound energy is applied at an MI of at least 0.8, 1.2, 1.4, 1.8, or 2.3. Aspects disclosed herein include a kit comprising: a linear double stranded DNA (dsDNA) construct covalently closed at each of its ends by DNA loops; a plurality of microbubbles; and instructions for administering to the subject, in proximity to a target cell(s) an effective amount of an ultrasound energy, sufficient to induce the formation of pores the target cell(s) by disrupting the plurality of microbubbles to result in introduction of the dsDNA into the target cell(s) and expression of the nucleic acid payload in the target cell(s).

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A provides an illustration of an example linear closed end double stranded DNA (dsDNA) construct;

FIG. 1B illustrates experimental protocols for experiments in which sonoporation gene therapy treatments comprised a dsDNA construct or a circular, double stranded miniplasmid DNA (mpDNA) DNA construct;

FIG. 2 illustrates gene expression of a luciferase transgene measured by IVIS fluorescence imaging showing average fluorescence radiance for subjects administered sonoporation gene therapy treatments with mpDNA DNA construct;

FIG. 3 illustrates gene expression of a luciferase transgene measured by IVIS fluorescence imaging showing average fluorescence radiance in subjects administered sonoporation gene therapy treatments with 500 μg of a mpDNA DNA construct (shown in FIG. 2, row 2);

FIG. 4A provides bar graphs illustrating gene expression of a luciferase transgene measured by IVIS fluorescence imaging showing average fluorescence radiance in subjects administered sonoporation gene therapy treatments with a mpDNA construct, in which a sustained decrease of nucleic acid payload expression at approximately 3 weeks after a sonoporation gene therapy treatment with the mpDNA construct is observed;

FIG. 4B provides bar graphs illustrating gene expression of a luciferase transgene measured by IVIS fluorescence imaging showing average fluorescence radiance in subjects administered sonoporation gene therapy treatments with a mpDNA construct, in which a sustained decrease of nucleic acid payload expression at approximately 3 weeks after sonoporation gene therapy treatment with the mpDNA construct is observed;

FIG. 4C provides a line plot illustrating gene expression of a luciferase transgene measured by IVIS fluorescence imaging showing average fluorescence radiance in subjects administered sonoporation gene therapy treatments with a mpDNA construct, in which a sustained decrease of nucleic acid payload expression at approximately 3 weeks after sonoporation gene therapy treatment with the mpDNA construct is observed;

FIG. 5 provides fluorescence images illustrating gene expression of a luciferase transgene measured by IVIS fluorescence imaging showing average fluorescence radiance in subjects administered sonoporation gene therapy treatments with a linear double stranded construct, in which it is illustrated there is sustained gene expression 11 weeks following the sonoporation gene therapy treatment with the linear double stranded construct;

FIG. 6A provides a bar plot illustrating gene expression of a luciferase transgene measured by IVIS fluorescence imaging showing average fluorescence radiance in subjects administered sonoporation gene therapy treatments with a linear double stranded construct, in which the durability of nucleic acid payload expression is shown up to one week after sonoporation gene therapy treatment with the linear double stranded construct;

FIG. 6B provides line plots illustrating gene expression of a luciferase transgene measured by IVIS fluorescence imaging showing average fluorescence radiance in subjects administered sonoporation gene therapy treatments with a linear double stranded construct, in which the durability of nucleic acid payload expression is shown at least five weeks after sonoporation gene therapy treatment with the linear double stranded construct;

FIG. 6C provides bar plots illustrating gene expression of a luciferase transgene measured by IVIS fluorescence imaging showing average fluorescence radiance in subjects administered sonoporation gene therapy treatments with a linear double stranded construct, in which the durability of nucleic acid payload expression is shown at least 14 weeks after sonoporation gene therapy treatment with the linear double stranded construct;

FIG. 7 shows ALT, IL6 and AST serum levels measured from subjects 24 hours following a sonoporation treatment, and illustrate that there was no elevation of ALT, IL6 and AST serum levels as a result of the sonoporation treatment;

FIG. 8A illustrates an embodiment of a sonoporation treatment at the cellular level;

FIG. 8B illustrates an embodiment of a sonoporation treatment at the cellular level;

FIG. 9 provides fluorescence images illustrating gene expression of a luciferase transgene measured by IVIS fluorescence imaging showing average fluorescence radiance in subjects administered sonoporation gene therapy treatments with a linear double stranded construct, in which it is illustrated there is sustained gene expression 12-26 weeks following the sonoporation gene therapy treatment with the linear double stranded construct;

FIG. 10 provides bar plots illustrating gene expression of a luciferase transgene measured by IVIS fluorescence imaging showing average fluorescence radiance in subjects administered sonoporation gene therapy treatments with a linear double stranded construct, in which the durability of nucleic acid payload expression is shown for at least 26 weeks after sonoporation gene therapy treatment with the linear double stranded construct;

FIG. 11 provides a bar plots illustrating gene expression of a luciferase transgene measured by IVIS fluorescence imaging showing average fluorescence radiance in subjects administered sonoporation gene therapy treatments with a linear double stranded construct as compared to a pDNA construct.

### DETAILED DESCRIPTION

One challenge appreciated by the inventors in sonoporation based gene therapy methods includes maintaining durable expression of the transgene in the weeks following transfection. For example, the use of conventional nucleotide constructs (e.g., plasmid DNA constructs) in sonoporation-mediated gene therapy can result in low transfection rates, undetectable levels of therapeutic protein expression, and/or in short-duration protein expression. It is appreciated by the inventors there remains a need in the art for improved processes which permit sonoporation gene therapy treatments that can improve gene transfection and gene expression of a nucleic acid payload in a cell over an extended period following a single sonoporation treatment. As such, disclosed herein are improved methods for sonoporation based gene therapy treatments which utilize next-generation DNA constructs, which increase gene transfection and expression, and which increase the durability of gene expression following sonoporation based gene therapy treatments. The methods disclosed herein can include administering a linear double stranded DNA (dsDNA) construct covalently closed at each of its ends by DNA loops, the linear dsDNA construct comprising the nucleic acid payload, the treatment resulting in sustained expression of a nucleotide payload. The methods of administering a linear double stranded DNA (dsDNA) construct to a subject using sonoporation can induce a steady state of gene expression for weeks following the sonoporation treatment under circumstances where expression of a payload encoded by a pDNA construct would fail to maintain sustained gene expression. The methods disclosed herein can further include administering treatment sessions at particular intervals and applying ultrasound energy using reduced power, reduced mechanical indexes, alternating between high and low mechanical indexes, in short flash pulses, and/or in short treatment sessions to achieve further beneficial results.

Provided herein are methods for nucleic acid transfection into and expression in a cell, tissue, or organ of a subject in a targeted manner using sonoporation (e.g., a process comprising applying an ultrasonic acoustic energy to a cell, tissue, or organ, such as to provide increased porosity in the cell, tissue, or organ). As used herein, in some embodiments, a subject herein is a mammal. In some embodiments, the mammal is, by way of non-limiting example, a human, a monkey, or another non-human primate. In some embodiments, the subject can be a rat, a mouse, or another non-primate animal.

Provided in certain embodiments herein are methods of inducing expression of a nucleic acid payload or protein in a target cell or tissue (e.g., of a subject), comprising administering the nucleic acid payload to the subject.

Provided in certain embodiments herein are methods of inducing expression of a nucleic acid payload or protein in a target cell or tissue (e.g., of a subject), comprising administering a plurality of microbubbles to the subject.

Provided in certain embodiments herein are methods of inducing expression of a nucleic acid payload or protein in a target cell or tissue (e.g., of a subject), comprising administering ultrasound energy to the subject in proximity to the target cell(s).

Provided in certain embodiments herein are methods of inducing expression of a nucleic acid payload in a target cell(s), organ(s), or tissue(s) (e.g., of a subject), comprising administering to the subject a linear double stranded DNA (dsDNA) construct covalently closed at each of its ends by DNA loops, the linear dsDNA construct comprising the nucleic acid payload. The methods of administering a linear double stranded DNA (dsDNA) construct to a subject using sonoporation can induce a steady state of gene expression for weeks following the sonoporation treatment under circumstances where expression of a payload encoded by a pDNA construct would fail to maintain sustained gene expression. In some embodiments, the method includes administering to the subject a dsDNA construct covalently closed at each of its ends by DNA loops. In some embodiments, the dsDNA construct covalently closed at each of its ends by DNA loops is an episomal nucleic acid delivery vector which does not integrate into the genome of the host cell. In some embodiments, dsDNA construct covalently closed at each of its ends by DNA loops behaves as an extrachromosomal elements in the nucleus of a transfected cell.

In some embodiments, a method provided herein comprises administering to the subject a plurality of sonoactive microstructures.

In some embodiments, a method provided herein comprises administering to the subject, in proximity to the target cell(s), organ(s), or tissue(s) an effective amount of an ultrasound energy, thereby inducing the formation of pores the target cell(s), organ(s), or tissue(s) by disrupting the plurality of microbubbles (e.g., sonoactive microstructures) to result in introduction of the dsDNA into the target cell(s), organ(s), or tissue(s) and expression of the nucleic acid payload in the target cell(s), organ(s), or tissue(s).

In some embodiments, a method provided herein comprises induction of expression of a nucleic acid payload within 3 hours. In some embodiments, a method provided herein comprises induction of expression of a nucleic acid payload within 4 hours. In some embodiments, a method provided herein comprises induction of expression of a nucleic acid payload within 5 hours. In some embodiments, a method provided herein comprises induction of expression of a nucleic acid payload within 6 hours. In some embodiments, a method provided herein comprises induction of expression of a nucleic acid payload within 12 hours. In some embodiments, a method provided herein comprises induction of expression of a nucleic acid payload within 18 hours. In some embodiments, a method provided herein comprises induction of expression of a nucleic acid payload within 24 hours.

Conditions under which sonoporation based gene therapies may be used to produce improved and durable gene expression in a subject which is superior to traditional gene therapy techniques are not clearly defined. Identifying methods for sonoporation in result in durable gene expression in a subject over an extended period would represent a significant benefit to patients. In some embodiments, when applying sonoporation treatments according to the methods disclosed herein, the expression of a protein encoded by the nucleic acid payload in the target cell(s) is maintained at a steady state at an elevated level as compared to a baseline expression level starting at least 3 weeks following performance of a method disclosed herein. In some embodiments, the steady state at the elevated level is within 1 order of magnitude of a peak level of expression. In some embodiments, the steady state is maintained for 4 weeks following a single sonoporation treatment. In some embodiments, the steady state is maintained for 5 weeks following a single sonoporation treatment. In some embodiments, the steady state is maintained for 6 weeks following a single sonoporation treatment. In some embodiments, the steady state is maintained for 7 weeks following a single sonoporation treatment. In some embodiments, the steady state is maintained for 8 weeks following a single sonoporation treatment. In some embodiments, the steady state is maintained for 9 weeks following a single sonoporation treatment. In some embodiments, the steady state is maintained for 10 weeks following a single sonoporation treatment. In some embodiments, the steady state is maintained for 11 weeks following a single sonoporation treatment. In some embodiments, the steady state is maintained for 12 weeks following a single sonoporation treatment. In some embodiments, the steady state is maintained for 13 weeks following a single sonoporation treatment. In some embodiments, the steady state is maintained for 14 weeks following a single sonoporation treatment. In some embodiments, the steady state is maintained for 15 weeks following a single sonoporation treatment. In some embodiments, the steady state is maintained for 16 weeks following a single sonoporation treatment. In some embodiments, the steady state is maintained for 17 weeks following a single sonoporation treatment. In some embodiments, the steady state is maintained for 18 weeks following a single sonoporation treatment. In some embodiments, the steady state is maintained for 19 weeks following a single sonoporation treatment. In some embodiments, the steady state is maintained for 20 weeks following a single sonoporation treatment. In some embodiments, the steady state is maintained for 21 weeks following a single sonoporation treatment. In some embodiments, the steady state is maintained for 22 weeks following a single sonoporation treatment. In some embodiments, the steady state is maintained for 23 weeks following a single sonoporation treatment. In some embodiments, the steady state is maintained for 24 weeks following a single sonoporation treatment. In some embodiments, the steady state is maintained for 25 weeks following a single sonoporation treatment. In some embodiments, the steady state is maintained for 26 weeks following a single sonoporation treatment.

In some embodiments, the linear dsDNA construct is administered at a first dosage that is less than a second dosage of a conventional plasmid (pDNA) construct comprising the nucleic acid payload under the same conditions required to induce an equivalent level of expression. In some embodiments, the first dosage is at least 10%, 20%, 30% 40%, 50%, 60% or 70%, less than the second dosage. In certain embodiments, the first dosage is 10% less than the second dosage. In certain embodiments, the first dosage is 20% less than the second dosage. In certain embodiments, the first dosage is 30% less than the second dosage. In certain embodiments, the first dosage is 40% less than the second dosage. In certain embodiments, the first dosage is 50% less than the second dosage. In certain embodiments, the first dosage is 60% less than the second dosage. In certain embodiments, the first dosage is 70% less than the second dosage.

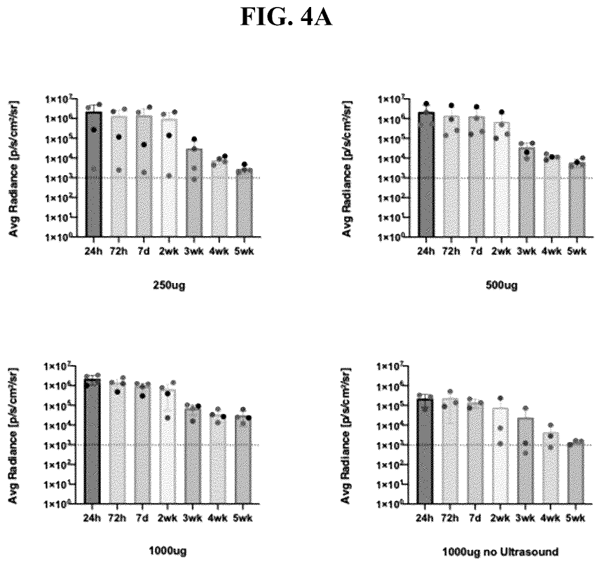

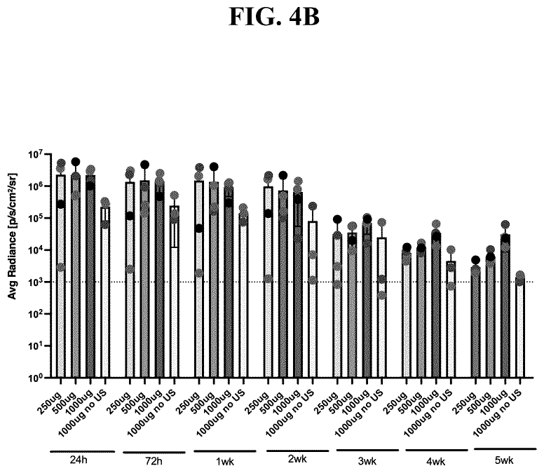

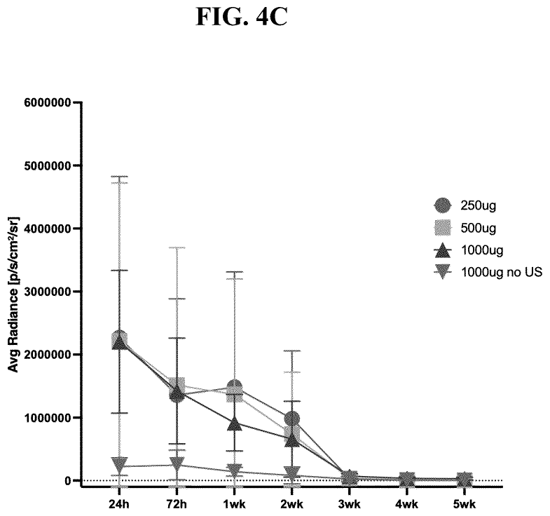

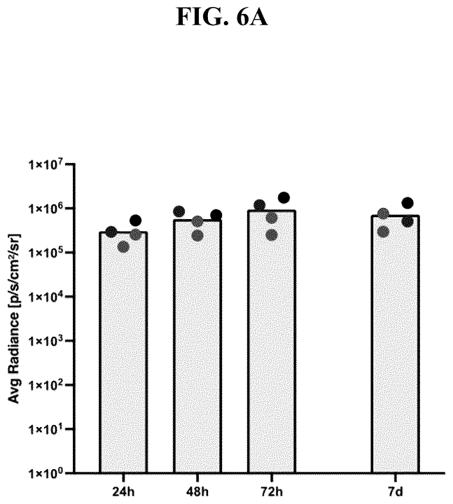

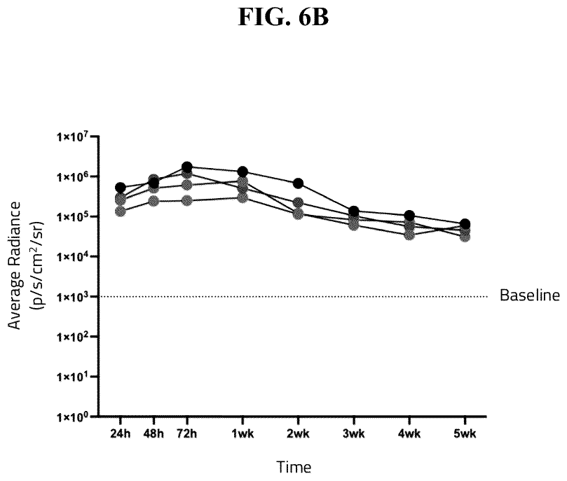

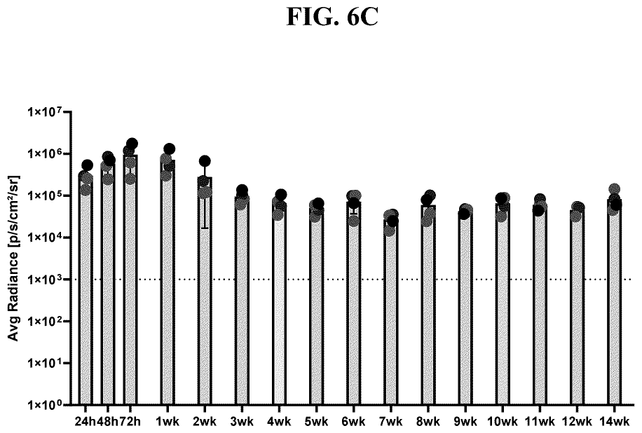

In some embodiments, the linear dsDNA construct is administered at a dosage that is the same as a dosage of a conventional plasmid (pDNA) construct comprising the nucleic acid payload under the same conditions required to induce an equivalent level of expression, with the gene expression being maintained for 1, 2, 3, 4, 5, 6, 7, 8, 9, 10, 11, 12, 13, 14, 15, 16, 17, 18, 19, 20, 21, 22, 23, 24, 25, or 26 weeks. In some embodiments, the linear dsDNA construct is administered at a dosage that is the same as a dosage of a conventional plasmid (pDNA) construct comprising the nucleic acid payload under the same conditions required to induce an equivalent level of expression, with the gene expression being maintained for 1, 2, 3, 4, 5, 6, 7, 8, 9, 10, 11, 12, 13, 14, 15, 16, 17, 18, 19, 20, 21, 22, 23, 24, 25, or 26 weeks after the dissipation of gene expression resulting from a plasmid (pDNA) construct. As is shown in FIGS. 4A-4C, the expression of the nucleic acid payload encoded by the pDNA construct failed to continue after about 3 weeks following the sonoporation treatment. In contrast, as is illustrated by FIGS. 6A-6C, the expression of the nucleic acid payload encoded by the linear double stranded DNA construct reached a steady state of ongoing expression at about 3 weeks following the sonoporation treatment, and maintained the steady state of ongoing expression for at least 14 weeks.

In some embodiments, the linear dsDNA construct is administered at a first dosage that is less than 500 micrograms (500 μg). In some embodiments, the linear dsDNA construct is administered at a first dosage that is less than 1.5 mg/kg bodyweight.

In some embodiments, the expression of a protein encoded by the nucleic acid payload in a target cell(s), organ(s), or tissue(s) is maintained for at least 1, 2, 3, 4, or 5, 6, 7, 8, 9, 10, 11, 12, 13, 14, 15, 16, 17, 18, 19, 20, 21, 22, 23, 24, 25, or 26 weeks. In some embodiments, the expression of a protein encoded by the nucleic acid payload in target cell(s), organ(s), or tissue(s) is maintained for at least 1 week. In some embodiments, the expression of a protein encoded by the nucleic acid payload in target cell(s), organ(s), or tissue(s) is maintained for at least 2 weeks. In some embodiments, the expression of a protein encoded by the nucleic acid payload in target cell(s), organ(s), or tissue(s) is maintained for at least 3 weeks. In some embodiments, the expression of a protein encoded by the nucleic acid payload in target cell(s), organ(s), or tissue(s) is maintained for at least 4 weeks. In some embodiments, the expression of a protein encoded by the nucleic acid payload in target cell(s), organ(s), or tissue(s) is maintained for at least 5 weeks. In some embodiments, the expression of a protein encoded by the nucleic acid payload in target cell(s), organ(s), or tissue(s) is maintained for at least 6 weeks. In some embodiments, the expression of a protein encoded by the nucleic acid payload in target cell(s), organ(s), or tissue(s) is maintained for at least 7 weeks. In some embodiments, the expression of a protein encoded by the nucleic acid payload in target cell(s), organ(s), or tissue(s) is maintained for at least 8 weeks. In some embodiments, the expression of a protein encoded by the nucleic acid payload in target cell(s), organ(s), or tissue(s) is maintained for at least 9 weeks. In some embodiments, the expression of a protein encoded by the nucleic acid payload in target cell(s), organ(s), or tissue(s) is maintained for at least 10 weeks. In some embodiments, the expression of a protein encoded by the nucleic acid payload in target cell(s), organ(s), or tissue(s) is maintained for at least 11 weeks. In some embodiments, the expression of a protein encoded by the nucleic acid payload in target cell(s), organ(s), or tissue(s) is maintained for at least 12 weeks. In some embodiments, the expression of a protein encoded by the nucleic acid payload in target cell(s), organ(s), or tissue(s) is maintained for at least 13 weeks. In some embodiments, the expression of a protein encoded by the nucleic acid payload in target cell(s), organ(s), or tissue(s) is maintained for at least 14 weeks. In some embodiments, the expression of a protein encoded by the nucleic acid payload in target cell(s), organ(s), or tissue(s) is maintained for at least 15 weeks. In some embodiments, the expression of a protein encoded by the nucleic acid payload in target cell(s), organ(s), or tissue(s) is maintained for at least 16 weeks. In some embodiments, the expression of a protein encoded by the nucleic acid payload in target cell(s), organ(s), or tissue(s) is maintained for at least 17 weeks. In some embodiments, the expression of a protein encoded by the nucleic acid payload in target cell(s), organ(s), or tissue(s) is maintained for at least 18 weeks. In some embodiments, the expression of a protein encoded by the nucleic acid payload in target cell(s), organ(s), or tissue(s) is maintained for at least 19 weeks. In some embodiments, the expression of a protein encoded by the nucleic acid payload in target cell(s), organ(s), or tissue(s) is maintained for at least 20 weeks. In some embodiments, the expression of a protein encoded by the nucleic acid payload in target cell(s), organ(s), or tissue(s) is maintained for at least 21 weeks. In some embodiments, the expression of a protein encoded by the nucleic acid payload in target cell(s), organ(s), or tissue(s) is maintained for at least 22 weeks. In some embodiments, the expression of a protein encoded by the nucleic acid payload in target cell(s), organ(s), or tissue(s) is maintained for at least 23 weeks. In some embodiments, the expression of a protein encoded by the nucleic acid payload in target cell(s), organ(s), or tissue(s) is maintained for at least 24 weeks. In some embodiments, the expression of a protein encoded by the nucleic acid payload in target cell(s), organ(s), or tissue(s) is maintained for at least 25 weeks. In some embodiments, the expression of a protein encoded by the nucleic acid payload in target cell(s), organ(s), or tissue(s) is maintained for at least 26 weeks.

In some embodiments, the expression of a protein encoded by the nucleic acid payload in a target cell(s), organ(s), or tissue(s) is maintained for at least 1, 2, 3, 4, 5, 6, 7, 8, 9, 10, 11, 12, 13, 14, 15, 16, 17, 18, 19, 20, 21, 22, 23, 24, 25, or 26 weeks following a single performance of a method described herein. In some embodiments, the expression of a protein encoded by the nucleic acid payload in a target cell(s), organ(s), or tissue(s) is maintained for at least 1 week following a single performance of a method described herein. In some embodiments, the expression of a protein encoded by the nucleic acid payload in a target cell(s), organ(s), or tissue(s) is maintained for at least 2 weeks following a single performance of a method described herein. In some embodiments, the expression of a protein encoded by the nucleic acid payload in a target cell(s), organ(s), or tissue(s) is maintained for at least 3 weeks following a single performance of a method described herein. In some embodiments, the expression of a protein encoded by the nucleic acid payload in a target cell(s), organ(s), or tissue(s) is maintained for at least 4 weeks following a single performance of a method described herein. In some embodiments, the expression of a protein encoded by the nucleic acid payload in a target cell(s), organ(s), or tissue(s) is maintained for at least 5 weeks following a single performance of a method described herein. In some embodiments, the expression of a protein encoded by the nucleic acid payload in a target cell(s), organ(s), or tissue(s) is maintained for at least 6 weeks following a single performance of a method described herein. In some embodiments, the expression of a protein encoded by the nucleic acid payload in a target cell(s), organ(s), or tissue(s) is maintained for at least 7 weeks following a single performance of a method described herein. In some embodiments, the expression of a protein encoded by the nucleic acid payload in a target cell(s), organ(s), or tissue(s) is maintained for at least 8 weeks following a single performance of a method described herein. In some embodiments, the expression of a protein encoded by the nucleic acid payload in a target cell(s), organ(s), or tissue(s) is maintained for at least 9 weeks following a single performance of a method described herein. In some embodiments, the expression of a protein encoded by the nucleic acid payload in a target cell(s), organ(s), or tissue(s) is maintained for at least 10 weeks following a single performance of a method described herein. In some embodiments, the expression of a protein encoded by the nucleic acid payload in a target cell(s), organ(s), or tissue(s) is maintained for at least 11 weeks following a single performance of a method described herein. In some embodiments, the expression of a protein encoded by the nucleic acid payload in a target cell(s), organ(s), or tissue(s) is maintained for at least 12 weeks following a single performance of a method described herein. In some embodiments, the expression of a protein encoded by the nucleic acid payload in a target cell(s), organ(s), or tissue(s) is maintained for at least 13 weeks following a single performance of a method described herein. In some embodiments, the expression of a protein encoded by the nucleic acid payload in a target cell(s), organ(s), or tissue(s) is maintained for at least 14 weeks following a single performance of a method described herein. In some embodiments, the expression of a protein encoded by the nucleic acid payload in a target cell(s), organ(s), or tissue(s) is maintained for at least 15 weeks following a single performance of a method described herein. In some embodiments, the expression of a protein encoded by the nucleic acid payload in a target cell(s), organ(s), or tissue(s) is maintained for at least 16 weeks following a single performance of a method described herein. In some embodiments, the expression of a protein encoded by the nucleic acid payload in a target cell(s), organ(s), or tissue(s) is maintained for at least 17 weeks following a single performance of a method described herein. In some embodiments, the expression of a protein encoded by the nucleic acid payload in a target cell(s), organ(s), or tissue(s) is maintained for at least 18 weeks following a single performance of a method described herein. In some embodiments, the expression of a protein encoded by the nucleic acid payload in a target cell(s), organ(s), or tissue(s) is maintained for at least 19 weeks following a single performance of a method described herein. In some embodiments, the expression of a protein encoded by the nucleic acid payload in a target cell(s), organ(s), or tissue(s) is maintained for at least 20 weeks following a single performance of a method described herein. In some embodiments, the expression of a protein encoded by the nucleic acid payload in a target cell(s), organ(s), or tissue(s) is maintained for at least 21 weeks following a single performance of a method described herein. In some embodiments, the expression of a protein encoded by the nucleic acid payload in a target cell(s), organ(s), or tissue(s) is maintained for at least 22 weeks following a single performance of a method described herein. In some embodiments, the expression of a protein encoded by the nucleic acid payload in a target cell(s), organ(s), or tissue(s) is maintained for at least 23 weeks following a single performance of a method described herein. In some embodiments, the expression of a protein encoded by the nucleic acid payload in a target cell(s), organ(s), or tissue(s) is maintained for at least 24 weeks following a single performance of a method described herein. In some embodiments, the expression of a protein encoded by the nucleic acid payload in a target cell(s), organ(s), or tissue(s) is maintained for at least 25 weeks following a single performance of a method described herein. In some embodiments, the expression of a protein encoded by the nucleic acid payload in a target cell(s), organ(s), or tissue(s) is maintained for at least 26 weeks following a single performance of a method described herein.

In some embodiments, the expression of a protein encoded by the nucleic acid payload in a target cell(s), organ(s), or tissue(s) is maintained at an elevated level as compared to a baseline expression level for at least 1, 2, 3, 4, 5, 6, 7, 8, 9, 10, 11, 12, 13, 14, 15, 16, 17, 18, 19, 20, 21, 22, 23, 24, 25, or 26 weeks following performance of a method described herein. In some embodiments, the expression of a protein encoded by the nucleic acid payload in a target cell(s), organ(s), or tissue(s) is maintained at an elevated level as compared to a baseline expression level for at least 1 week following performance of a method described herein. In some embodiments, the expression of a protein encoded by the nucleic acid payload in a target cell(s), organ(s), or tissue(s) is maintained at an elevated level as compared to a baseline expression level for at least 2 weeks following performance of a method described herein. In some embodiments, the expression of a protein encoded by the nucleic acid payload in a target cell(s), organ(s), or tissue(s) is maintained at an elevated level as compared to a baseline expression level for at least 3 weeks following performance of a method described herein. In some embodiments, the expression of a protein encoded by the nucleic acid payload in a target cell(s), organ(s), or tissue(s) is maintained at an elevated level as compared to a baseline expression level for at least 4 weeks following performance of a method described herein. In some embodiments, the expression of a protein encoded by the nucleic acid payload in a target cell(s), organ(s), or tissue(s) is maintained at an elevated level as compared to a baseline expression level for at least 5 weeks following performance of a method described herein. In some embodiments, the expression of a protein encoded by the nucleic acid payload in a target cell(s), organ(s), or tissue(s) is maintained at an elevated level as compared to a baseline expression level for at least 6 weeks following performance of a method described herein. In some embodiments, the expression of a protein encoded by the nucleic acid payload in a target cell(s), organ(s), or tissue(s) is maintained at an elevated level as compared to a baseline expression level for at least 7 weeks following performance of a method described herein. In some embodiments, the expression of a protein encoded by the nucleic acid payload in a target cell(s), organ(s), or tissue(s) is maintained at an elevated level as compared to a baseline expression level for at least 8 weeks following performance of a method described herein. In some embodiments, the expression of a protein encoded by the nucleic acid payload in a target cell(s), organ(s), or tissue(s) is maintained at an elevated level as compared to a baseline expression level for at least 9 weeks following performance of a method described herein. In some embodiments, the expression of a protein encoded by the nucleic acid payload in a target cell(s), organ(s), or tissue(s) is maintained at an elevated level as compared to a baseline expression level for at least 10 weeks following performance of a method described herein. In some embodiments, the expression of a protein encoded by the nucleic acid payload in a target cell(s), organ(s), or tissue(s) is maintained at an elevated level as compared to a baseline expression level for at least 11 weeks following performance of a method described herein. In some embodiments, the expression of a protein encoded by the nucleic acid payload in a target cell(s), organ(s), or tissue(s) is maintained at an elevated level as compared to a baseline expression level for at least 12 weeks following performance of a method described herein. In some embodiments, the expression of a protein encoded by the nucleic acid payload in a target cell(s), organ(s), or tissue(s) is maintained at an elevated level as compared to a baseline expression level for at least 13 weeks following performance of a method described herein. In some embodiments, the expression of a protein encoded by the nucleic acid payload in a target cell(s), organ(s), or tissue(s) is maintained at an elevated level as compared to a baseline expression level for at least 14 weeks following performance of a method described herein. In some embodiments, the expression of a protein encoded by the nucleic acid payload in a target cell(s), organ(s), or tissue(s) is maintained at an elevated level as compared to a baseline expression level for at least 15 weeks following performance of a method described herein. In some embodiments, the expression of a protein encoded by the nucleic acid payload in a target cell(s), organ(s), or tissue(s) is maintained at an elevated level as compared to a baseline expression level for at least 16 weeks following performance of a method described herein. In some embodiments, the expression of a protein encoded by the nucleic acid payload in a target cell(s), organ(s), or tissue(s) is maintained at an elevated level as compared to a baseline expression level for at least 17 weeks following performance of a method described herein. In some embodiments, the expression of a protein encoded by the nucleic acid payload in a target cell(s), organ(s), or tissue(s) is maintained at an elevated level as compared to a baseline expression level for at least 18 weeks following performance of a method described herein. In some embodiments, the expression of a protein encoded by the nucleic acid payload in a target cell(s), organ(s), or tissue(s) is maintained at an elevated level as compared to a baseline expression level for at least 19 weeks following performance of a method described herein. In some embodiments, the expression of a protein encoded by the nucleic acid payload in a target cell(s), organ(s), or tissue(s) is maintained at an elevated level as compared to a baseline expression level for at least 20 weeks following performance of a method described herein. In some embodiments, the expression of a protein encoded by the nucleic acid payload in a target cell(s), organ(s), or tissue(s) is maintained at an elevated level as compared to a baseline expression level for at least 21 weeks following performance of a method described herein. In some embodiments, the expression of a protein encoded by the nucleic acid payload in a target cell(s), organ(s), or tissue(s) is maintained at an elevated level as compared to a baseline expression level for at least 22 weeks following performance of a method described herein. In some embodiments, the expression of a protein encoded by the nucleic acid payload in a target cell(s), organ(s), or tissue(s) is maintained at an elevated level as compared to a baseline expression level for at least 23 weeks following performance of a method described herein. In some embodiments, the expression of a protein encoded by the nucleic acid payload in a target cell(s), organ(s), or tissue(s) is maintained at an elevated level as compared to a baseline expression level for at least 24 weeks following performance of a method described herein. In some embodiments, the expression of a protein encoded by the nucleic acid payload in a target cell(s), organ(s), or tissue(s) is maintained at an elevated level as compared to a baseline expression level for at least 25 weeks following performance of a method described herein. In some embodiments, the expression of a protein encoded by the nucleic acid payload in a target cell(s), organ(s), or tissue(s) is maintained at an elevated level as compared to a baseline expression level for at least 26 weeks following performance of a method described herein.

In some embodiments, inducing expression of the nucleic acid payload comprises inducing production of a nucleotide sequence encoded by the payload. In some embodiments, inducing expression of the nucleic acid payload comprises inducing production of RNA encoded by the payload. In some embodiments, inducing expression of the nucleic acid payload comprises inducing production of messenger RNA (mRNA) encoded by the payload. In some embodiments, inducing expression of the nucleic acid payload comprises inducing production of small interfering RNA (siRNA) encoded by the payload. In some embodiments, inducing expression of the nucleic acid payload comprises inducing production of small hairpin RNA (shRNA) encoded by the payload.

In some embodiments, the expression of a nucleotide sequence encoded by the nucleic acid payload in a target cell(s), organ(s), or tissue(s) is maintained for at least 1, 2, 3, 4, or 5, 6, 7, 8, 9, 10, 11, 12, 13, 14, 15, 16, 17, 18, 19, 20, 21, 22, 23, 24, 25, or 26 weeks. In some embodiments, the expression of a nucleotide sequence encoded by the nucleic acid payload in target cell(s), organ(s), or tissue(s) is maintained for at least 1 week. In some embodiments, the expression of a nucleotide sequence encoded by the nucleic acid payload in target cell(s), organ(s), or tissue(s) is maintained for at least 2 weeks. In some embodiments, the expression of a nucleotide sequence encoded by the nucleic acid payload in target cell(s), organ(s), or tissue(s) is maintained for at least 3 weeks. In some embodiments, the expression of a nucleotide sequence encoded by the nucleic acid payload in target cell(s), organ(s), or tissue(s) is maintained for at least 4 weeks. In some embodiments, the expression of a nucleotide sequence encoded by the nucleic acid payload in target cell(s), organ(s), or tissue(s) is maintained for at least 5 weeks. In some embodiments, the expression of a nucleotide sequence encoded by the nucleic acid payload in target cell(s), organ(s), or tissue(s) is maintained for at least 6 weeks. In some embodiments, the expression of a nucleotide sequence encoded by the nucleic acid payload in target cell(s), organ(s), or tissue(s) is maintained for at least 7 weeks. In some embodiments, the expression of a nucleotide sequence encoded by the nucleic acid payload in target cell(s), organ(s), or tissue(s) is maintained for at least 8 weeks. In some embodiments, the expression of a nucleotide sequence encoded by the nucleic acid payload in target cell(s), organ(s), or tissue(s) is maintained for at least 9 weeks. In some embodiments, the expression of a nucleotide sequence encoded by the nucleic acid payload in target cell(s), organ(s), or tissue(s) is maintained for at least 10 weeks. In some embodiments, the expression of a nucleotide sequence encoded by the nucleic acid payload in target cell(s), organ(s), or tissue(s) is maintained for at least 11 weeks. In some embodiments, the expression of a nucleotide sequence encoded by the nucleic acid payload in target cell(s), organ(s), or tissue(s) is maintained for at least 12 weeks. In some embodiments, the expression of a nucleotide sequence encoded by the nucleic acid payload in target cell(s), organ(s), or tissue(s) is maintained for at least 13 weeks. In some embodiments, the expression of a nucleotide sequence encoded by the nucleic acid payload in target cell(s), organ(s), or tissue(s) is maintained for at least 14 weeks. In some embodiments, the expression of a nucleotide sequence encoded by the nucleic acid payload in target cell(s), organ(s), or tissue(s) is maintained for at least 15 weeks. In some embodiments, the expression of a nucleotide sequence encoded by the nucleic acid payload in target cell(s), organ(s), or tissue(s) is maintained for at least 16 weeks. In some embodiments, the expression of a nucleotide sequence encoded by the nucleic acid payload in target cell(s), organ(s), or tissue(s) is maintained for at least 17 weeks. In some embodiments, the expression of a nucleotide sequence encoded by the nucleic acid payload in target cell(s), organ(s), or tissue(s) is maintained for at least 18 weeks. In some embodiments, the expression of a nucleotide sequence encoded by the nucleic acid payload in target cell(s), organ(s), or tissue(s) is maintained for at least 19 weeks. In some embodiments, the expression of a nucleotide sequence encoded by the nucleic acid payload in target cell(s), organ(s), or tissue(s) is maintained for at least 20 weeks. In some embodiments, the expression of a nucleotide sequence encoded by the nucleic acid payload in target cell(s), organ(s), or tissue(s) is maintained for at least 21 weeks. In some embodiments, the expression of a nucleotide sequence encoded by the nucleic acid payload in target cell(s), organ(s), or tissue(s) is maintained for at least 22 weeks. In some embodiments, the expression of a nucleotide sequence encoded by the nucleic acid payload in target cell(s), organ(s), or tissue(s) is maintained for at least 23 weeks. In some embodiments, the expression of a nucleotide sequence encoded by the nucleic acid payload in target cell(s), organ(s), or tissue(s) is maintained for at least 24 weeks. In some embodiments, the expression of a nucleotide sequence encoded by the nucleic acid payload in target cell(s), organ(s), or tissue(s) is maintained for at least 25 weeks. In some embodiments, the expression of a nucleotide sequence encoded by the nucleic acid payload in target cell(s), organ(s), or tissue(s) is maintained for at least 26 weeks.

In some embodiments, the expression of a nucleotide sequence encoded by the nucleic acid payload in a target cell(s), organ(s), or tissue(s) is maintained for at least 1, 2, 3, 4, 5, 6, 7, 8, 9, 10, 11, 12, 13, 14, 15, 16, 17, 18, 19, 20, 21, 22, 23, 24, 25, or 26 weeks following a single performance of a method described herein. In some embodiments, the expression of a nucleotide sequence encoded by the nucleic acid payload in a target cell(s), organ(s), or tissue(s) is maintained for at least 1 week following a single performance of a method described herein. In some embodiments, the expression of a nucleotide sequence encoded by the nucleic acid payload in a target cell(s), organ(s), or tissue(s) is maintained for at least 2 weeks following a single performance of a method described herein. In some embodiments, the expression of a nucleotide sequence encoded by the nucleic acid payload in a target cell(s), organ(s), or tissue(s) is maintained for at least 3 weeks following a single performance of a method described herein. In some embodiments, the expression of a nucleotide sequence encoded by the nucleic acid payload in a target cell(s), organ(s), or tissue(s) is maintained for at least 4 weeks following a single performance of a method described herein. In some embodiments, the expression of a nucleotide sequence encoded by the nucleic acid payload in a target cell(s), organ(s), or tissue(s) is maintained for at least 5 weeks following a single performance of a method described herein. In some embodiments, the expression of a nucleotide sequence encoded by the nucleic acid payload in a target cell(s), organ(s), or tissue(s) is maintained for at least 6 weeks following a single performance of a method described herein. In some embodiments, the expression of a nucleotide sequence encoded by the nucleic acid payload in a target cell(s), organ(s), or tissue(s) is maintained for at least 7 weeks following a single performance of a method described herein. In some embodiments, the expression of a nucleotide sequence encoded by the nucleic acid payload in a target cell(s), organ(s), or tissue(s) is maintained for at least 8 weeks following a single performance of a method described herein. In some embodiments, the expression of a nucleotide sequence encoded by the nucleic acid payload in a target cell(s), organ(s), or tissue(s) is maintained for at least 9 weeks following a single performance of a method described herein. In some embodiments, the expression of a nucleotide sequence encoded by the nucleic acid payload in a target cell(s), organ(s), or tissue(s) is maintained for at least 10 weeks following a single performance of a method described herein. In some embodiments, the expression of a nucleotide sequence encoded by the nucleic acid payload in a target cell(s), organ(s), or tissue(s) is maintained for at least 11 weeks following a single performance of a method described herein. In some embodiments, the expression of a nucleotide sequence encoded by the nucleic acid payload in a target cell(s), organ(s), or tissue(s) is maintained for at least 12 weeks following a single performance of a method described herein. In some embodiments, the expression of a nucleotide sequence encoded by the nucleic acid payload in a target cell(s), organ(s), or tissue(s) is maintained for at least 13 weeks following a single performance of a method described herein. In some embodiments, the expression of a nucleotide sequence encoded by the nucleic acid payload in a target cell(s), organ(s), or tissue(s) is maintained for at least 14 weeks following a single performance of a method described herein. In some embodiments, the expression of a nucleotide sequence encoded by the nucleic acid payload in a target cell(s), organ(s), or tissue(s) is maintained for at least 15 weeks following a single performance of a method described herein. In some embodiments, the expression of a nucleotide sequence encoded by the nucleic acid payload in a target cell(s), organ(s), or tissue(s) is maintained for at least 16 weeks following a single performance of a method described herein. In some embodiments, the expression of a nucleotide sequence encoded by the nucleic acid payload in a target cell(s), organ(s), or tissue(s) is maintained for at least 17 weeks following a single performance of a method described herein. In some embodiments, the expression of a nucleotide sequence encoded by the nucleic acid payload in a target cell(s), organ(s), or tissue(s) is maintained for at least 18 weeks following a single performance of a method described herein. In some embodiments, the expression of a nucleotide sequence encoded by the nucleic acid payload in a target cell(s), organ(s), or tissue(s) is maintained for at least 19 weeks following a single performance of a method described herein. In some embodiments, the expression of a nucleotide sequence encoded by the nucleic acid payload in a target cell(s), organ(s), or tissue(s) is maintained for at least 20 weeks following a single performance of a method described herein. In some embodiments, the expression of a nucleotide sequence encoded by the nucleic acid payload in a target cell(s), organ(s), or tissue(s) is maintained for at least 21 weeks following a single performance of a method described herein. In some embodiments, the expression of a nucleotide sequence encoded by the nucleic acid payload in a target cell(s), organ(s), or tissue(s) is maintained for at least 22 weeks following a single performance of a method described herein. In some embodiments, the expression of a nucleotide sequence encoded by the nucleic acid payload in a target cell(s), organ(s), or tissue(s) is maintained for at least 24 weeks following a single performance of a method described herein. In some embodiments, the expression of a nucleotide sequence encoded by the nucleic acid payload in a target cell(s), organ(s), or tissue(s) is maintained for at least 14 weeks following a single performance of a method described herein. In some embodiments, the expression of a nucleotide sequence encoded by the nucleic acid payload in a target cell(s), organ(s), or tissue(s) is maintained for at least 26 weeks following a single performance of a method described herein. In some embodiments, the expression of a nucleotide sequence encoded by the nucleic acid payload in a target cell(s), organ(s), or tissue(s) is maintained for at least 14 weeks following a single performance of a method described herein.

In some embodiments, the expression of a nucleotide sequence encoded by the nucleic acid payload in a target cell(s), organ(s), or tissue(s) is maintained at an elevated level as compared to a baseline expression level for at least 1, 2, 3, 4, 5, 6, 7, 8, 9, 10, 11, 12, 13, 14, 15, 16, 17, 18, 19, 20, 21, 22, 23, 24, 25, or 26 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 1 week following performance of a method described herein. In some embodiments, the expression of a nucleotide sequence encoded by the nucleic acid payload in a target cell(s), organ(s), or tissue(s) is maintained at an elevated level as compared to a baseline expression level for at least 2 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 3 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 4 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 5 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 6 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 7 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 8 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 9 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 10 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 11 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 12 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 13 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 14 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 15 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 16 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 17 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 18 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 19 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 20 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 21 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 22 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 23 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 24 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 25 weeks following performance of a method described herein. In some embodiments, the expression of a nucleotide sequence encoded by the nucleic acid payload in a target cell(s), organ(s), or tissue(s) is maintained at an elevated level as compared to a baseline expression level for at least 26 weeks following performance of a method described herein.

In preparing ultrasound protocols to be utilized in repeated sonoporation treatment sessions, there is significant ambiguity within the art as to the identification of ultrasound parameters which are safe for use repeated sonoporation treatment sessions, so as to improve gene transfection and expression, without inducing inflammation of the target organ, tissue, or cells; cellular, damage, inflammation, or death; or otherwise inducing elevation of inflammatory or apoptotic cellular biomarker within the subject. Aspects disclosed herein provide methods of repeating sonoporation gene therapy treatments without resulting in cellular, damage, inflammation, or death; or otherwise inducing elevation of inflammatory or apoptotic cellular biomarker within the subject. In some embodiments, administering an effective amount of ultrasound energy can comprise continuously applying ultrasound energy for the duration of a treatment session. In some embodiments, administering an effective amount of ultrasound energy comprises continuously applying ultrasound energy for a portion of the duration of the treatment session. In some embodiments, ultrasound energy can be applied continuously. In some more some embodiments, ultrasound energy can be continuously applied by continuously alternating between ultrasound energy at a first mechanical index applied for a first duration and ultrasound energy at a second mechanical index applied for a second duration. In some embodiments, the ultrasound energy can be applied at alternating mechanical indices without ceasing application of the ultrasound energy. In some embodiments, ultrasound energy can be applied by applying ultrasound energy at a first mechanical index for a first duration, applying ultrasound energy at a second mechanical index for a second duration, and re-applying ultrasound energy at the first mechanical index without ceasing application of ultrasound energy for the duration of a treatment session.

As used herein, an ultrasound “flash” refers to application of ultrasound energy at a different or second mechanical index compared to an initial or a first application of ultrasound energy. In some embodiments, the application of ultrasound energy comprises administering a plurality of ultrasound flashes in which the ultrasound energy is applied at a high mechanical index for a brief duration before returning to application of the ultrasound energy at the lower mechanical index. In some embodiments, these ultrasound flashes can be several seconds apart from one another. In some embodiments, ultrasound energy is administered at the first mechanical index or the initial mechanical index in between application of the ultrasound flashes. In some embodiments, an ultrasound flash can have a mechanical index which is increased relative to a first mechanical index. In some embodiments, administering ultrasound energy comprises administering at least five ultrasound flashes at a second mechanical index that is increased relative to a first mechanical index with an interval between.

In some embodiments, the ultrasound energy is applied at a mechanical index ranging from 0.05 to 2.3. In some embodiments, the ultrasound energy is applied at a mechanical index ranging from 0.05 to 1.8. In some embodiments, the ultrasound energy is applied at any suitable mechanical index. In some embodiments, the ultrasound energy is applied at any suitable mechanical index for disrupting the microbubbles and facilitating entry of the payload into the target cell(s). In some embodiments, the ultrasound energy is applied at lower mechanical index, and a higher mechanical index, continuously, in alternating pulses. In some embodiments, the lower mechanical index ranges from 0.05 to about 1.5. In some embodiments, the higher mechanical index ranges from 1.6 to about 2.3. In some embodiments, the alternating pulses occur in intervals ranging from 0.9 μs (microseconds) to 10 s. In some embodiments, an ultrasound flash may have a mechanical index which is increased relative to a first mechanical index. In some embodiments, administering ultrasound energy comprises administering at least four ultrasound flashes at a mechanical index that is increased relative to a first mechanical index with an interval between flashes of less than 10 seconds. In some embodiments, administering ultrasound energy comprises administering ultrasound flashes with a duration of about 0.9 us to about 2.5 us. In some embodiments, the administering the effective amount of the ultrasound energy comprises administering at least 5 ultrasound flashes at a second mechanical index which is increased relative to a first mechanical index, the ultrasound flashes at the second mechanical index being administered less than 10 seconds apart from one another. In some embodiments, the administering the effective amount of the ultrasound energy comprises administering a plurality of ultrasound flashes at an increased mechanical index, the ultrasound flashes at the increased mechanical index being administered about 1 s to about 10 s apart from one another. In some embodiments, the administering the effective amount of the ultrasound energy comprises administering a plurality of ultrasound flashes at an increased mechanical index, with each of the plurality of ultrasound flashes at the increased mechanical index being administered about 5 s to about 10 s apart from one another. In some embodiments, the administering the effective amount of the ultrasound energy comprises administering a plurality of ultrasound flashes at an increased mechanical index, the ultrasound flashes at the increased mechanical index being administered 1 s to about 5 s apart from one another. In some embodiments, the ultrasound flash(s) is an application of ultrasound at the increased mechanical index or the second mechanical index over a time period. In some embodiments, the time period is less than 1 s. In some embodiments, the time period is from about 0.7 us to about 3 us (microseconds). In some embodiments, the time period is about 0.72, 0.82, 0.72, 0.98, or 2.28 us. In some embodiments, the administering of the ultrasound energy of c. comprises administering at least 5 ultrasound flashes at the second mechanical index.

In some embodiments, the ultrasound energy is applied for up to 30, 60, 90, 120, 150, 180, 240, 300, or 360 1200 seconds. In some embodiments, the ultrasound energy is applied for up to 30 seconds. In some embodiments, the ultrasound energy is applied for up to 60 seconds. In some embodiments, the ultrasound energy is applied for up to 90 seconds. In some embodiments, the ultrasound energy is applied for up to 120 seconds. In some embodiments, the ultrasound energy is applied for up to 150 seconds. In some embodiments, the ultrasound energy is applied for up to 180 seconds. In some embodiments, the ultrasound energy is applied for up to 240 seconds. In some embodiments, the ultrasound energy is applied for up to 300 seconds. In some embodiments, the ultrasound energy is applied for up to 360 seconds.

In some embodiments, the treatment session as determined by the length of continuous application of ultrasound energy is up to 30, 60, 90, 120, 150, 180, 240, 300, or 360 seconds. In some embodiments, the treatment session as determined by the length of continuous application of ultrasound energy is up to 30 seconds. In some embodiments, the treatment session as determined by the length of continuous application of ultrasound energy is up to 60 seconds. In some embodiments, the treatment session as determined by the length of continuous application of ultrasound energy is up to 90 seconds. In some embodiments, the treatment session as determined by the length of continuous application of ultrasound energy is up to 120 seconds. In some embodiments, the treatment session as determined by the length of continuous application of ultrasound energy is up to 150 seconds. In some embodiments, the treatment session as determined by the length of continuous application of ultrasound energy is up to 180 seconds. In some embodiments, the treatment session as determined by the length of continuous application of ultrasound energy is up to 240 seconds. In some embodiments, the treatment session as determined by the length of continuous application of ultrasound energy is up to 300 seconds. In some embodiments, the treatment session as determined by the length of continuous application of ultrasound energy is up to 360 seconds.

In some embodiments, the treatment session as determined by the length of continuous application of ultrasound energy is up to 5, 10, 15, 20, 25, 30, 35, or 40 minutes. In some embodiments, the treatment session as determined by the length of continuous application of ultrasound energy is up to 5 min. In some embodiments, the treatment session as determined by the length of continuous application of ultrasound energy is up to 10 min. In some embodiments, the treatment session as determined by the length of continuous application of ultrasound energy is up to 15 min. In some embodiments, the treatment session as determined by the length of continuous application of ultrasound energy is up to 20 min. In some embodiments, the treatment session as determined by the length of continuous application of ultrasound energy is up to 25 min. In some embodiments, the treatment session as determined by the length of continuous application of ultrasound energy is up to 30 min. In some embodiments, the treatment session as determined by the length of continuous application of ultrasound energy is up to 35 min. In some embodiments, the treatment session as determined by the length of continuous application of ultrasound energy is up to 40 min.

Undesirable effects on living cells or tissues can occur due to ultrasound applications. In some embodiments, the present disclosure provides methods for improvement of gene transfection that does not result in substantial DNA or cell damage in the target cells, tissues, or organs. In some embodiments, the method does not result in substantial cellular damage to the target cell. In some embodiments, the method does not result in inflammation of the target tissue, or in the elevation of apoptotic or inflammatory biomarkers. Cellular damage can be detected using apoptotic biomarkers. For example, in the liver, detection of released hepatocellular transaminases, e.g., serum alanine aminotransferase (ALT) or aspartate aminotransferase (AST), can be an indicator of apoptotic hepatocytes. Additional apoptotic biomarkers can comprise interleukin 6 (IL6) or B-cell lymphoma 2 (BCL2 or BCL2 apoptosis regulator). In some embodiments, Biomarkers of cellular damage, inflammation, or death are not detected at apoptotic levels during or in a time period following application of the sonoporation treatment methods disclosed herein. In some embodiments, biomarkers of cell damage are not detected at clinical elevated levels during or in a time period immediately following application of the method disclosed herein. In some embodiments, a method disclosed herein does not result in a significant elevation of inflammatory biomarker. Non-limiting examples of markers of cellular damage, inflammation, or apoptosis include ALT, AST, IL6, and BCL2. In some embodiments, a method disclosed herein does not result in a significant, clinically significant, other elevation of ALT, AST, IL6, or BCL2.

In some embodiments, the method comprises administering an effective amount of ultrasound energy transcutaneously to the subject. In some embodiments, the method comprises administering an effective amount of ultrasound energy transcutaneously to the subject in proximity to target cell(s). In some embodiments, the target cell(s) are hepatic cell(s). In some embodiments, the target cell(s) are renal cell(s). In some embodiments, the target cell(s) are pancreatic cell(s). In some embodiments, the target cell(s) are comprised in a tissue. In some embodiments, the target tissue is comprised in an organ. In some embodiments, the organ is the liver. In some embodiments, the organ is a kidney. In some embodiments, the organ is the pancreas. In some embodiments, after administering of the nucleic acid construct and sonoactive microstructures, the ultrasound acoustic energy is applied at the target cell, tissue, or organ. In some embodiments, the ultrasound acoustic energy is applied at the target cell, tissue, or organ during administration of the nucleic acid construct, the sonoactive microstructures or both. In some embodiments, the ultrasound acoustic energy is applied at the target cell, tissue, or organ during and after administration of the nucleic acid construct, the sonoactive microstructures or both.

In some embodiments, the nucleic acid payload comprises a nonendogenous gene. In some embodiments, the transgene comprises a detectible marker. In some embodiments, the transgene comprises luciferase. In some embodiments, inducing expression of the nucleic acid payload comprises inducing expression of luciferase. In some embodiments, the nucleic acid payload is configured to perform gene augmentation, gene replacement, or gene editing. In some embodiments, the nucleic acid payload can be a therapeutic payload. In some embodiments, the nucleic acid payload comprises a therapeutic transgene. In some embodiments, the nucleic acid construct is a miniplasmid comprising a nucleic acid payload. In some embodiments, the nucleic acid payload comprises a transgene. In some embodiments, the transgene can be an endogenous transgene. In some embodiments, the transgene can be a non-endogenous transgene. In some embodiments, the transgene comprises a therapeutic transgene. In some embodiments, the therapeutic transgene comprises: GLP-1, INS, Reg3g, MafA, PDX-1, NUEROG3, NGN3, DRYK, DYRK1A, DYRK1B, Factor VIII, Factor IX, or combinations thereof. In some embodiments, the therapeutic transgene comprises GLP-1. In some embodiments, the therapeutic transgene comprises INS. In some embodiments, the therapeutic transgene comprises REg3g. In some embodiments, the therapeutic transgene comprises MafA. In some embodiments, the therapeutic transgene comprises PDX-1. In some embodiments, the therapeutic transgene comprises NUEROG3. In some embodiments, the therapeutic transgene comprises NGN3. In some embodiments, the therapeutic transgene comprises DYRK1A. In some embodiments, the therapeutic transgene comprises DYRK1B. In some embodiments, the therapeutic transgene comprises Factor VIII. In some embodiments, the therapeutic transgene comprises Factor IX.

In some embodiments, the nucleic acid payload comprises a therapeutic transgene coupled to a promoter. In some embodiments, a nucleic acid payload comprises a regulatory element such as a promoter, (e.g., APOE-ATT). In some embodiments, the nucleic acid payload comprises a therapeutic transgene coupled to a promoter other than CMV promoter or ubiquitin C (UbC) promoter. In some embodiments, the nucleic acid construct comprises a regulatory element such as a promoter, enhancer, ribosome binding site, or transcription termination signal. Examples of promoters contemplated herein include, but are not limited to, e.g., CMV promoter, UbC promoter, CAG promoter, EF1-α promoter, ApoE promoter, AAT-promoter, ApoE-AAT1 promoter, 3XSERP promoter, P3-hybrid promoter, or combinations thereof. In some embodiments, the promoter comprises an ApoE promoter. In some embodiments, the promoter comprises a CAG promoter. In some embodiments, the promoter comprises an AAT promoter.

In some embodiments, the nucleic acid payload and the plurality of microbubbles are administered in a volumetric ratio of at least 1-part nucleic acid payload solution to 4 parts microbubble solution. In some embodiments, the nucleic acid payload and the plurality of microbubbles are administered in a volumetric ratio of about 1-part nucleic acid payload solution to 3 parts microbubble solution. In some embodiments, the nucleic acid payload and the plurality of microbubbles are administered in a volumetric ratio of about 1-part nucleic acid payload solution to 2 parts microbubble solution. In some embodiments, the nucleic acid payload and the plurality of microbubbles are administered in a volumetric ratio of about 1-part nucleic acid payload solution to 1 part microbubble solution.

In some embodiments, the administering of the nucleic acid construct and the sonoactive microstructures is by intravenous administration. In some embodiments, the intravenous administration is through a peripheral vein. In some embodiments, a peripheral vein is any vein outside of the torso. Exemplary peripheral veins include, but are not limited to, the jugular vein, the brachiocephalic veins, the saphenous vein, and veins of the forearm, head, and feet. Benefits of peripheral vein administration result in lower hydrostatic pressure and levels of peak negative pressure within the target tissue/organ as compared to other methods of administration, for example portal vein or inferior vena cava injections. Peripheral vein injections can promote serial injections within using invasive access, and can promote the methods of repeating sonoporation disclosed herein. However, administration through a peripheral vein may not result in delivery, or in substantially reduced delivery, of the microbubbles or nucleic acid payload to the target tissue or cells. When administering the nucleic acid payload and microbubbles intravenously through a peripheral vein using the methods disclosed herein, sonoporation treatment sessions can be repeated without resulting in substantial cellular damage, inflammation, and/or death following the treatment session. When administering the nucleic acid payload and microbubbles intravenously through a peripheral vein in a treatment session using the methods disclosed herein, transfection of nucleic acid payload to a cell can be increased, and expression of the nucleic acid or amino acid sequence encoded by the nucleic acid payload may be increased.

In some embodiments, inducing expression of the nucleic acid payload comprises inducing production of RNA encoded by the payload. In some embodiments, inducing expression of the nucleic acid payload comprises inducing production of protein encoded by the payload. In some embodiments, regulating expression of the nucleic acid payload comprises regulating production of RNA encoded by the payload. In some embodiments, regulating expression of the nucleic acid payload comprises regulating production of protein encoded by the nucleic acid payload, or regulating production of protein encoded by an mRNA encoded by the nucleic acid payload.

The methods described herein can be used to treat a subject in need of gene therapy or enzyme replacement treatment. In some respects, the present disclosure provides methods of treating a subject having a liver condition. In some embodiments, the liver condition treated is: Wilson's Disease, Cholestasis progressive familial intrahepatic, Von Willebrand disease, Hemophilia A, Hemophilia B, Factor 5 deficiency, Alpha-Mannosidosis, Gaucher disease (glucocerebrosidase deficiency, glucocerebrosidosis), Niemann Pick Disease A/B, Carbamoylphosphate Synthetase I Deficiency, Glycogen Storage Disease Type III, Cystinosis, A1AT deficiency, Citrullinemia Type I & II. In some embodiments, the liver condition is treated by transfection of a therapeutic transgene, where the condition and therapeutic transgene (e.g., one or more of the transgenes) are: Wilson's Disease: ATP7B (1465AA); Cholestasis, progressive familial intrahepatic (PFIC1-4): ABCB11 (1321AA), ABCB4 (1286AA), ATP8B1 (1251AA), TJP2 (1190AA); Von Willebrand Disease: VWF (2813AA); Hemophilia A: FVIII (2351AA); Hemophilia B: FIX (415AA); Factor V Deficiency: F5 (2224AA); Alpha-Mannosidosis: MAN2B1 (1011AA); Gaucher disease (glucocerebrosidase deficiency, glucocerebrosidosis): GBA (536AA); Niemann Pick Disease A/B: SMPD1 (631AA); Carbamoylphosphate Synthetase I Deficiency: CPS1 (1500AA); Glycogen Storage Disease Type III: GDE/AGL (1532AA); Cystinosis: CTNS (367AA); A1AT deficiency: SERPINA1 (418AA); Citrullinemia Type I & II: ASS1 (412AA), SLC25A13 (675AA).

In one aspect, using the sonoporation methods described herein, the present disclosure provides methods of treating a subject having a kidney condition. In some embodiments, the kidney condition treated is acute kidney injury (AKI), Alport Syndrome, or Autosomal Dominant Polycystic Kidney Disease. In some embodiments, the kidney condition is treated by transfection of a therapeutic transgene, where the condition and therapeutic transgene are (e.g., one or more of the transgenes): Alport Syndrome (COL4A3, COL4A4, COL4A5); Autosomal Dominant Polycystic Kidney Disease (PKD1, PKD2).

In one aspect, using the sonoporation methods described herein, the present disclosure provides methods of treating a subject having a kidney condition. In some embodiments, the kidney condition treated is: Alport Syndrome, or Autosomal Dominant Polycystic Kidney Disease. In some embodiments, the kidney condition is treated by transfection of a therapeutic transgene, where the therapeutic transgene is (e.g., one or more of the transgenes): COL4A4; COL4A5; PKD1; PKD2. In some embodiments, the kidney condition is treated by transfection of a therapeutic transgene, where the condition and therapeutic transgene (e.g., one or more of the transgenes) are: Alport Syndrome (COL4A3, COL4A4, COL4A5); Autosomal Dominant Polycystic Kidney Disease (PKD1, PKD2).

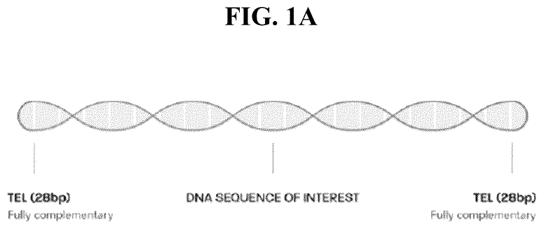

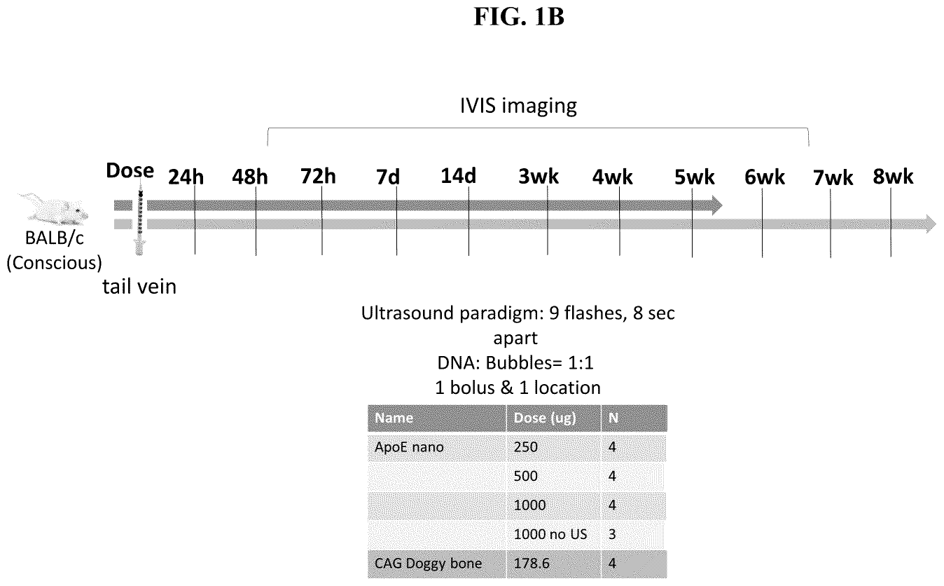

Double stranded DNA nucleic acid (dsDNA) constructs can be utilized to deliver a transgene or a nonendogenous gene to cells in target cell-types, tissues, or organs. In some embodiments, the dsDNA construct is a linear dsDNA construct. In some embodiments, the dsDNA construct is a doggy bone DNA™ (dbDNA™ construct, FIG. 1A, Touchlight Genetics Ltd., UK). In some embodiments, the linear dsDNA construct comprises a protelomerase recognition sequence in each loop. In some embodiments, the protelomerase recognition sequence comprises at least 14 nucleotides of a double stranded palindromic sequence. In some embodiments, the linear dsDNA construct comprises at least one eukaryotic promoter. In some embodiments, the linear dsDNA construct comprises at least one stem loop motif comprising a central non-complementary loop section flanked by two complementary sequences. In some embodiments, the loops are hairpin loops.

In some embodiments, the nucleic acid construct is a miniplasmid. As used herein, the term “miniplasmid (mpDNA)” refers to circular nucleic acid constructs that are smaller in size (i.e., contain fewer base pairs (bp)) than conventional plasmids or pDNA, with regard to the size of the nucleic acid construct excluding the therapeutic transgene, promoters, and regulatory elements. In some embodiments, mpDNA constructs are smaller than 500 bp. In some embodiments, mpDNA constructs are smaller than 600 bp. As used herein, the term “Nanoplasmid™” (e.g., Nanoplasmid sourced from Aldevron, Fargo, South Dakota.) refers to a small mpDNA construct that comprises a plasmid backbone that is less than 500 bp and does not comprise an antibiotic resistance gene. In some embodiments, the nucleic acid payload comprises a HALO DNA construct. In some embodiments, the nucleic acid payload comprises a double stranded, covalently closed, linear DNA construct. In some embodiments, the nucleic acid payload comprises a DNA construct comprising structural elements facilitating nuclear entry. In some embodiments, the nucleic acid payload comprises a DNA construct comprising structural elements facilitating durable gene expression following gene transfection. Miniplasmid DNA nucleic acid constructs can be utilized to deliver a transgene or a nonendogenous gene to cells in target cell-types, tissues, or organs. In some embodiments, the mpDNA construct comprises less than 500 base pairs excluding a transgene. In some embodiments, the mpDNA construct does not comprise antibiotic resistant genes. In some embodiments, the mpDNA construct does not comprise nucleotide sequences that encode bacterial genes. In some embodiments, the mpDNA construct does not comprise a bacterial genome. In some embodiments, the mpDNA construct comprises a therapeutic transgene and/or a regulatory element. In some embodiments, the mpDNA construct is a Nanoplasmid™.

In some embodiments, a total amount of DNA administered to a subject for purposes of sonoporation can range from about 1 micrograms (μg) to about 200 mg. In some embodiments, a total amount of DNA administered to a subject is 20 mg to 100 mg. In some embodiments, a total amount of DNA administered to a subject is about 20 mg. In some embodiments, a total amount of DNA administered to a subject is about 50 mg.

In some embodiments, the nucleic acid construct is administered at a dosage of up to about 0.2 mg/kg bodyweight. In some embodiments, the nucleic acid construct is administered at a dosage between 0.4 mg/kg bodyweight to 1.5 mg/kg bodyweight. In some embodiments, the nucleic acid construct is administered at a concentration of about 3.5 μg/μl.

Sonoactive microstructures (also referred to as microbubbles) contemplated herein include, but are not limited to, those used as ultrasonic imaging contrast agents. In some embodiments, the sonoactive microstructures comprise a phospholipid stabilized microstructure. In some embodiments, the sonoactive microstructures comprise a phospholipid stabilized shell. In some embodiments, the sonoactive microstructures comprise a lipid stabilized shell. In some embodiments, the sonoactive microstructures comprise a protein stabilized shell. In some embodiments, the sonoactive microstructures comprise an albumin stabilized shell. In some embodiments, the phospholipid stabilized microstructure comprises a high molecular weight gas core, e.g., a perflutren core. Examples of sonoactive microstructures include, but are not limited to, OPTISON (GE Healthcare), Sonazoid (GE Healthcare), or DEFINITY and Definity RT (Lantheus Medical Imaging, Inc). In some embodiments, the sonoactive microstructures are Sonazoid microbubbles, Definity microbubbles, or Definity RT microbubbles. In some embodiments, the sonoactive microstructures are Optison microbubbles.

The sonoactive microstructures can be administered prior to, after, or simultaneous (e.g., co-administered) with the administration of the nucleic acid construct (or nucleic acid payload). In some embodiments, the nucleic acid construct and the sonoactive microstructures are coadministered. In some embodiments, the administering of the nucleic acid construct and the sonoactive microstructures occurs serially, concurrently, sequentially, or continuously. In some embodiments, the administering of the nucleic acid construct and the sonoactive microstructures occurs serially. In some embodiments, the administering of the nucleic acid construct and the sonoactive microstructures occurs concurrently. In some embodiments, the administering of the nucleic acid construct and the sonoactive microstructures occur sequentially. In some embodiments, the administering of the nucleic acid construct and the sonoactive microstructures occurs during the course of the ultrasound treatment continuously (e.g., is administered intravenously throughout).

In some embodiments, the sonoactive microstructures are administered at a dosage of about 1-50 ml, for example, 1 ml of Optison. The sonoactive microstructures can be administered at a concentration of about 500 M (million) to about 800 M microstructures per ml. In some embodiments, the sonoactive microstructures are administered at a concentration of about 5*10\hat{ }8 to about 1.2*10\hat{ }10 microstructures/ml, for example, 1*10\hat{ }9 microstructure/ml of Definity RT. In some embodiments, the concentration of microbubbles administered is up to about 1.2*10\hat{ }10 microbubbles/ml. In some embodiments, the concentration of microbubbles administered is at least 5*10\hat{ }8. In some embodiments, the sonoactive microstructures are administered at a dose of about 0.1 to about 0.8 mg microstructures/kg of body mass. In some embodiments, the sonoactive microstructures are administered at a dose of about 0.1 to about 1.0 ml/kg of body mass. In some embodiments, the sonoactive microstructures are administered at a concentration of about 10\hat{ }9 microstructures/ml. In some embodiments, the sonoactive microstructures are administered at a concentration of about 5*10\hat{ }8 to about 8*10\hat{ }8 microstructures/ml.

In some embodiments, the nucleic acid construct and the sonoactive microstructures are mixed prior to being coadministered. In some instances, the sonoactive microstructures are mixed with the nucleic acid constructs along with additional buffers or agents such as saline or other biocompatible solutions with varying electrostatic charges, surface chemistries, and ligands before administering to the subject. For example, Optison sonoactive microstructures can be mixed with a nanoplasmid and saline and administered together.

In some embodiments, the nucleic acid construct and the sonoactive microstructures are mixed prior to being coadministered. In some instances, the sonoactive microstructures are mixed with the nucleic acid constructs along with additional buffers or agents such as saline or other biocompatible solutions with varying electrostatic charges, surface chemistries, and ligands before administering to the subject. For example, Optison sonoactive microstructures can be mixed with a nanoplasmid and saline and administered together.

Provided herein are methods for nucleic acid transfection into and expression in a cell, tissue, or organ of a subject in a targeted manner using sonoporation (e.g., a process comprising applying an ultrasonic acoustic energy to a cell, tissue, or organ, such as to provide increased porosity in the cell, tissue, or organ). In one aspect, the present disclosure provides methods for delivery of the nucleic acid payload to a target cell by optimizing parameters or protocols of applied ultrasonic acoustic energy, including methods for increasing or decreasing expression of a gene in a target cell by applying ultrasonic acoustic energy at alternating mechanical indexes to induce stable vibration cavitation, and inertial cavitation of the sonoactive microstructures. In some cases, the nucleic acid payload is a miniplasmid, and delivery of the alternating mechanical indexes to induce stable vibration cavitation, and inertial cavitation of the sonoactive microstructures enhances delivery of the miniplasmid to the target cell.

Provided in certain embodiments herein are methods for transfecting a nucleic acid construct into a target cell or tissue (e.g., of a subject) by applying a first ultrasonic acoustic energy to a cell, tissue, or organ, and applying a second ultrasonic acoustic energy to the cell, tissue, or organ. In specific embodiments herein are methods for transfecting a nucleic acid construct into a target cell or tissue by applying a first ultrasonic acoustic energy having a first mechanical index (MI) and applying a second ultrasonic acoustic energy having a second mechanical index (MI). The present disclosure provides methods for enhancing transfection of a nucleic acid construct into the target cell or tissue by applying alternating ultrasonic acoustic energy, the alternating acoustic energy alternating between a first mechanical index (MI) and a second MI. Application of ultrasonic acoustic energy can be repeated several times during sonoporation, such as to increase the efficiency of nucleic acid construct transfection and/or delivery.

In some embodiments, a process provided herein provides sonoporation at two or more different ultrasonic acoustic energies (e.g., a first and second ultrasonic acoustic energy having a first and second MI, respectively). In certain embodiments, a process provided herein provides a process wherein an ultrasonic acoustic energy is continuously applied (e.g., ultrasonic acoustic energy transitions from the first ultrasonic acoustic energy to the second ultrasonic acoustic energy, without a period of no ultrasonic acoustic energy being applied). In certain embodiments, a transitory (e.g., third, fourth, etc.) ultrasonic acoustic energy is applied between application of the first and second ultrasonic acoustic energies.

In some embodiments, a sonoporation treatment (e.g., application of a first ultrasonic acoustic energy, a second ultrasonic acoustic energy, a single cycle of a first ultrasonic acoustic energy and a second ultrasonic acoustic energy, or series of cycles comprising a plurality of applications of a first ultrasonic acoustic energy and a plurality of applications of a second acoustic energy) can last for a few seconds (e.g., 1-100 seconds) or more, such as up to a few minutes (e.g., 1-3 minutes). In specific embodiments, a sonoporation treatment lasts for 1-30 seconds. In some specific embodiments, a sonoporation treatment lasts for 5-100 seconds. In certain embodiments, a sonoporation treatment lasts for at least 1 minute (e.g., 1-30 minutes).

In some embodiments, a first MI is a Low MI (e.g., less than 0.4). In certain embodiments, a second MI is a High MI (e.g., 0.4 or greater). In some embodiments, a first MI is a Low MI (e.g., less than 0.4) and a second MI is a High MI (e.g., 0.4 or greater). In some embodiments, a second MI is a Low MI (e.g., less than 0.4). In certain embodiments, a first MI is a High MI (e.g., 0.4 or greater). In specific embodiments, a second MI is a Low MI (e.g., less than 0.4) and a first MI is a High MI (e.g., 0.4 or greater).

In some embodiments, a Low MI is <0.3. In specific embodiments, a Low MI is <0.2. In more specific embodiments, a Low MI is <0.1. In still more specific embodiments, a Low MI is about 0.09. In still more specific embodiments, a Low MI is about 0.04. In still more specific embodiments, a Low MI is about 0.03.

In some embodiments, a High MI is >0.5. In specific embodiments, a High MI is 0.5 to 2.0 or is between 0.5 and 2.0. In more specific embodiments, a High MI is 0.5 to 1 or is between 0.5 and 2.0. In some embodiments, a High MI is 1.5. In some embodiments, a High MI is 1.8. In some embodiments, a High MI is 2.0. In some embodiments, a High MI is greater than 0.4. In some embodiments, a High MI is >0.5. In more specific embodiments, a High MI is 0.5 to 1 or is between 0.5 and 2.0. In some embodiments, a High MI is 1.5. In some embodiments, a High MI is 1.8. In some embodiments, a High MI is 2.0.

In certain embodiments, any process provided herein (e.g., a sonoporation treatment) comprises administering of a continuous ultrasonic acoustic energy (which may have varying energy levels) that alternates (e.g., in identical, similar, or variable periods) between Low MI and High MI. In some embodiments, a low MI (e.g., <0.1) (e.g., first) ultrasonic acoustic energy (also referred to herein as a Low MI) is administered to the subject, and a set number pulses (e.g., of less than 30 seconds) of High MI (e.g., second) ultrasonic acoustic energy (also referred to herein as a High MI) is administered to the subject. In some embodiments, a process provided herein comprises administration of a plurality of pulses of high MI (e.g., second) ultrasonic acoustic energy, e.g., during an otherwise continuous administration of a low MI (e.g., first) ultrasonic acoustic energy. In specific embodiments, the number of High MI pulses is about 4 or more, such as up to about 12, or an unlimited number of pulses. In specific embodiments the number of High MI pulses is 6-30. In still more specific embodiments, the number of High MI pulses is between 8, 9, 12, 15, or 18, or any number therebetween. In some embodiments, at least 8, 9, 12, 15, or 18 high MI pulses are administered to the subject in between applications of low MI ultrasound acoustic energy.

In some embodiments, high MI ultrasound acoustic energy is administered in a pulse. In specific embodiments, a pulse length is any suitable length, such as less than 30 seconds. In more specific embodiments, a pulse length is less than 15 seconds. In still more specific embodiments, a pulse length is less than 10 seconds. In yet more specific embodiments, a pulse length is less than 5 seconds. In more specific embodiments, a pulse length is less than 2 seconds. In still more specific embodiments, a pulse length is less than 1 second and/or may be greater than or equal to 1 microsecond. In some embodiments, a pulse length ranges from 100 to 300 microseconds. In some embodiments, a pulse length is up to about 200 microseconds. In some embodiments, a pulse length is up to about 500 microseconds. In some embodiments, a pulse length ranges from 1 to 500 microseconds.

In various embodiments, a High MI ultrasonic acoustic energy is provided first temporally (e.g., first in order). In other embodiments, a Low MI ultrasonic acoustic energy is provided second temporally (e.g., second in order).

In some embodiments, any process provided herein further comprises administering (e.g., systemically administering, such as via infusion) a nucleic acid (e.g., any nucleic acid provided herein) to a subject (e.g., to whom the ultrasonic acoustic energies are applied).

In some embodiments, any process provided herein further comprises administering (e.g., systemically administering, such as via infusion) a sonoactive structure (e.g., any sonoactive structure or microbubble described herein) to a subject (e.g., to whom the ultrasonic acoustic energies are applied).

In certain embodiments, provided herein is a method of delivering a nucleic acid payload in a target cell (e.g., of a tissue or organ) of a subject, the method comprising: (a) administering to the subject a nucleic acid construct comprising the nucleic acid payload; (b) administering to the subject a plurality of sonoactive microstructures; and (c) administering a sonoporation treatment.

In some embodiments, the sonoporation treatment comprises applying an ultrasonic acoustic energy to the target cell (e.g., of a tissue or organ of the subject) (e.g., the ultrasonic acoustic energy having a mechanical index (MI)). In some embodiments, applying an ultrasonic acoustic energy to the target cell comprises applying a first ultrasonic acoustic energy to the target cell and applying a second ultrasonic acoustic energy to the target cell. In some embodiments, the (e.g., first or second) ultrasonic acoustic energy has a first mechanical index (MI). In certain embodiments, (e.g., the other of the first or second) ultrasonic energy has a second mechanical index (MI). In some embodiments, the (e.g., first or second) MI is less than 0.4. In certain embodiments (e.g., the other of the first or second) MI is greater than 0.4 (e.g., and less than 2.0).

In specific embodiments, a first ultrasonic acoustic energy and a second ultrasonic acoustic energy are applied sequentially in a repeated manner.

In certain embodiments, the first (either High MI or Low MI) ultrasonic acoustic energy is applied before or after administration of any other agent, such as the nucleic acid and/or sonoactive structure. In some embodiments, the first ultrasonic acoustic energy is applied after administration of the sonoactive structure to the subject. In certain embodiments, the first ultrasonic acoustic energy is applied after administration of the nucleic acid to the subject. In some embodiments, the first ultrasonic acoustic energy is applied after administration of both the nucleic acid and the sonoactive structure(s).

In some embodiments, the first ultrasonic acoustic energy is administered within 60 minutes of administration of the nucleic acid and/or sonoactive structure(s). In specific embodiments, the first ultrasonic acoustic energy is administered within 30 minutes of administration of the nucleic acid and/or sonoactive structure(s). In more specific embodiments, the first ultrasonic acoustic energy is administered within 5 minutes of administration of the nucleic acid and/or sonoactive structure(s). In still more specific embodiments, the first ultrasonic acoustic energy is administered within 2 minutes of administration of the nucleic acid and/or sonoactive structure(s). In still more specific embodiments, the first ultrasonic acoustic energy may be applied simultaneously with administration of the nucleic acid and/or sonoactive structure(s).

In specific embodiments, the first (e.g., High MI) ultrasonic acoustic energy is applied immediately upon administration (e.g., infusion) or a period of time after administration (e.g., infusion) of the sonoactive structure(s) and/or nucleic acid.

In some embodiments, either the first or second ultrasonic acoustic energy is an ultrasonic acoustic energy (e.g., Low MI) that when applied to a cell, tissue, or organ of a subject results in stable cavitation (or stable vibrational cavitation) of the sonoactive structure and/or a change in the average diameter of the sonoactive structure(s), for example, due to inherent resonance properties of the microbubbles.

In certain embodiments, the first or second ultrasonic acoustic energy is an ultrasonic acoustic energy (e.g., High MI) that when applied to a cell, tissue, or organ of a subject results in inertial cavitation or the collapse of the sonoactive structures and/or disruption of cell membrane and/or vascular endothelial integrity.

In certain embodiments, either the first or second ultrasonic acoustic energy is an ultrasonic acoustic energy (e.g., Low MI) that when applied to a cell, tissue, or organ of a subject results in stable cavitation (or stable vibrational cavitation) and/or a change in the average diameter of the sonoactive structure(s), and the other of the first or second ultrasonic acoustic energy is an ultrasonic acoustic energy (e.g., High MI) that when applied to a cell, tissue, or organ of a subject results in inertial cavitation or the collapse of the sonoactive structures and/or disruption of cell membrane and/or vascular endothelial integrity.

In some instances, disruption of cell membrane allows target cells to become permeable to circulating agents such as nucleic acid constructs. In certain instances, such circulating agents can then enter the target cells, tissues or organs, such as in a more rapid manner (e.g., relative to either Low MI or High MI ultrasonic acoustic energy application alone, or in the absence of ultrasonic acoustic energy application).

In some embodiments, the methods herein comprise alternating the ultrasonic acoustic energy applied between a first ultrasonic acoustic energy having a first MI and a second ultrasonic acoustic energy having a second MI. In some embodiments, applying alternating ultrasonic acoustic energy administered to a subject between a first MI and a second MI is performed repeatedly over a number of times, such as to enhance gene transfection into the target cells, tissue or organ (e.g., relative to a similar process wherein a first and second ultrasonic acoustic energy are not used and/or are not alternately applied and/or are not alternately applied repeatedly).

In some embodiments, the method comprises administering ultrasound energy transcutaneously to the subject in proximity to one or more target cells. In some embodiments, the one or more target cells are hepatic cells. In some embodiments, the one or more target cells are renal cells. In some embodiments, the one or more target cells are pancreatic cells. In some embodiments, the one or more target cells are cardiac cells. In some embodiments, the one or more target cells are myocytes. In some embodiments, the one or more target cells are neuronal cells. In some embodiments, the one or more target cells are brain cells. In some embodiments, the one or more target cells are blood cells (e.g., white blood cells). In some embodiments, the target cells are cancerous cells.

In some embodiments, the one or more target cells are comprised in a tissue. In some embodiments, the tissue is skeletal muscle tissue. In some embodiments, the tissue is smooth muscle tissue. In some embodiments, the tissue is connective tissue. In some embodiments, the tissue is lymphatic tissue. In some embodiments, the tissue is nervous tissue. In some embodiments, the tissue is diseased tissue, e.g., cancerous tissue, fibrotic tissue, or tissue otherwise in need of gene therapy.

In some embodiments, the target tissue is comprised in an organ. In some embodiments, the organ is the liver. In some embodiments, the organ is a kidney. In some embodiments, the organ is the pancreas. In some embodiments, the organ is the heart. In some embodiments, the organ is the brain. In some embodiments, the one or more target cells are comprised in a tumor. In some embodiments, the tumor is a solid tumor. In some embodiments, the tumor is a liquid tumor.

In some embodiments, cells, tissue or organ are those of the liver. In some embodiments, cells, tissue or organ are those of the kidney.

In certain embodiments, a subject herein is a mammal. In some embodiments, the mammal is, by way of non-limiting example, a human, rat, mouse, monkey, and other non-human primates.

In certain embodiments, changing parameters of the ultrasound acoustic energy or MI can be performed to induce and/or enhance an expression of a transgene in a cell or an organ of a subject. In one aspect, provided herein are methods of transfection by alternating the ultrasonic acoustic energy using a first MI and a second MI. In some embodiments, the first MI that results in stable vibrational cavitation is applied prior to the second MI, which results in inertial cavitation. In some embodiments, the ultrasonic acoustic energy using the first MI and the second MI are reapplied for a number of times to increase transfection efficiency at the target cell. In some embodiments, during the application of sonoporation, the ultrasonic acoustic energy is applied at the first MI continuously except for when the ultrasonic acoustic energy is applied at the second MI. For example, applying an ultrasonic acoustic energy to the target cell at the first MI then applying an ultrasonic acoustic energy to the target cell at the second MI are repeated between 4 to 18 times. In some embodiments, applying an ultrasonic acoustic energy to the target cell at the first MI then applying an ultrasonic acoustic energy to the target cell at the second MI are repeated an unlimited number of times. In one aspect, during this time, the ultrasonic acoustic energy of the first MI is applied continuously except for when the ultrasonic acoustic energy of the second MI is applied.

In some embodiments, the first MI ranges from about 0.05 to about 0.4. In some embodiments, the first MI ranges from about 0.05 to about 0.3. In some embodiments, the first MI ranges from about 0.05 to about 0.4. In some embodiments, the first MI ranges from about 0.09 to about 0.3.

In some embodiments, the second MI ranges from about 0.5 to about 2.0. In some embodiments, the second MI ranges from greater than 1.4 to about 1.8. In some embodiments, the second MI ranges from greater than 1.4 to about 2.0. In some embodiments, the second MI ranges from about 1.5 to about 2.0.

In some embodiments, applying the ultrasonic acoustic energy at the first MI, and the applying the ultrasonic acoustic energy at the second MI are repeated between 4 and 18 times. In some embodiments, applying the ultrasonic acoustic energy at the first MI, and the applying the ultrasonic acoustic energy at the second MI are repeated between 6 and 12 times. In some embodiments, applying the ultrasonic acoustic energy at the first MI, and the applying the ultrasonic acoustic energy at the second MI are repeated between 8 and 10 times. In some embodiments, applying the ultrasonic acoustic energy at the first MI, and the applying the ultrasonic acoustic energy at the second MI are repeated between 8 and 18 times. In some embodiments, applying the ultrasonic acoustic energy at the first MI, and the applying the ultrasonic acoustic energy at the second MI are repeated 9 times.

In some embodiments, the applying the ultrasound acoustic energy comprises applying the ultrasonic acoustic energy at the first MI, and the applying the ultrasonic acoustic energy at the second MI, without ceasing applying the ultrasonic acoustic energy at the first MI, and the applying the ultrasonic acoustic energy at the second MI. In some embodiments, the applying the ultrasound acoustic energy comprises applying the ultrasonic acoustic energy at the first MI except for when the ultrasonic acoustic energy is applied at the second MI. In some embodiments, an ultrasound probe applying the ultrasonic acoustic energy is in constant contact with the surface of the subject's skin at the location of application (e.g., abdomen, chest wall, skull, etc.). In some embodiments, an ultrasound transducer that applies the ultrasonic acoustic energy to the target cell is continuously in contact with tissue of the subject and is continuously either (1) applying the ultrasound acoustic energy to the subject or (2) receiving reflected ultrasound energy from the subject. In certain embodiments, a transitory (e.g., third, fourth, etc.) ultrasonic acoustic energy is applied between application of the first and second ultrasonic acoustic energies. In certain embodiments, applying the ultrasound acoustic energy comprises applying the ultrasonic acoustic energy without regard to an EKG gating signal regulating the application of the ultrasound acoustic energy. In certain embodiments, applying the ultrasound acoustic energy comprises applying the ultrasonic acoustic energy without turning off power to the ultrasound transducer off. In some embodiments, applying the ultrasound acoustic energy comprises an ultrasound transducer sending ultrasound acoustic energy or receiving reflected ultrasound acoustic energy at least 95% of a period of time in which an ultrasound transducer continuously is contacting the subject.

In some instances, the ultrasonic acoustic energy of the second MI (e.g., high MI) is applied using a pulse. In some instances, a pulse comprises applying the ultrasonic acoustic energy in a short pulse (e.g., microsecond length pulse). In some cases, the high MI is applied with the pulse, results in induces inertial cavitation and destruction of the sonoactive microstructure, resulting in the disruption of cell membrane and vascular endothelial integrity, transducing the nucleic acid payload to the cell. In some instances, the pulse is applied with a duration of about 1 μs to about 200 μs. In some instances, the pulse is applied with a duration of about 1 μs to about 200 μs or greater.

In some embodiments, applying the ultrasonic acoustic energy at the second MI comprises applying the ultrasonic acoustic energy at the second MI using a pulse. In some instances, the duration of the second MI applied ranges from 0.1 μs to about 200 μs. In some instances, the duration of the second MI applied ranges from 1 μs to about 200 μs or greater. In some embodiments, applying the ultrasonic acoustic energy at the second MI comprises applying the ultrasonic acoustic energy at the second MI using a pulse with a duration of about 1 μs to about 200 μs. In some embodiments, applying the ultrasonic acoustic energy at the second MI comprises applying the ultrasonic acoustic energy at the second MI using a pulse with a duration of up to 200 μs. In some embodiments, applying the ultrasonic acoustic energy at the second MI comprises applying the ultrasonic acoustic energy at the second MI using a pulse with a duration of about 1 μs to about 500 μs. In some embodiments, applying the ultrasonic acoustic energy at the second MI comprises applying the ultrasonic acoustic energy at the second MI using a pulse with a duration of up to 500 μs. In some embodiments, applying the ultrasonic acoustic energy at the second MI comprises applying the ultrasonic acoustic energy at the second MI using a pulse with a duration of about 2.3 μs. In some embodiments, applying the ultrasonic acoustic energy at the second MI comprises applying the ultrasonic acoustic energy at the second MI using a pulse with a duration at least 2.3 μs. In some embodiments, applying the ultrasonic acoustic energy at the second MI comprises applying the ultrasonic acoustic energy at the second MI using a pulse with a duration ranging from 1-500 μs. In some embodiments, applying the ultrasonic acoustic energy at the second MI comprises applying the ultrasonic acoustic energy at the second MI using a pulse with a duration ranging from 0.1-500 μs.

In some cases, alternating the ultrasonic acoustic energy between the first MI and the second MI for a number of times also allows reperfusion of the sonoactive microstructures and the nucleic acid constructs to the target cell, tissue, or organ, following disruption of the sonoactive microstructures within or proximal to the target cell, tissue, or organ.

In some embodiments, the repeating application of ultrasonic acoustic energy between the first MI and the second MI comprises applying the ultrasonic acoustic energy at the first MI for an amount of time sufficient to permit reperfusion of the sonoactive microstructures in a tissue comprising the target cell before reapplying the ultrasonic acoustic energy at the second MI.

In some embodiments, the method comprises applying the ultrasonic acoustic energy at the first MI for 1-30 seconds before repeating the applying the ultrasound acoustic energy of (d). In some embodiments, the method comprises applying the ultrasonic acoustic energy at the first MI for 5-15 seconds before repeating the applying the ultrasound acoustic energy of reapplying the ultrasonic acoustic energy at the second MI. In some embodiments, the method comprises applying the ultrasonic acoustic energy at the first MI for 10 seconds before repeating the applying the ultrasound acoustic energy of reapplying the ultrasonic acoustic energy at the second MI.

In some instances, the duration of the first MI applied ranges from about 2 s to about 30 s. In some embodiments, applying the ultrasonic acoustic energy at the first MI comprises initially applying the ultrasonic acoustic energy at the first MI from about 2 s to about 30 s.

In some embodiments, applying the ultrasonic acoustic energy at the first MI, and the applying the ultrasonic acoustic energy at the second MI are repeated for a total amount of time ranging from about 1 s to about 60 m. In some embodiments, applying the ultrasonic acoustic energy at the first MI, and the applying the ultrasonic acoustic energy at the second MI are repeated for a total amount of time ranging from about 60 s to about 120 s.

In some embodiments, applying the ultrasonic acoustic energy at the first MI induces stable vibration cavitation of the sonoactive microstructures. In some embodiments, applying the ultrasonic acoustic energy at the first MI does not induce substantial disruption of the sonoactive microstructures. In some embodiments, applying the ultrasonic acoustic energy at the first MI does not induce substantial disruption of the sonoactive microstructures in a vasculature space and an extravascular space, or induces stable vibration cavitation of the sonoactive microstructures in a vasculature space and an extravascular space.

In some embodiments, applying the ultrasonic acoustic energy at the first MI induces formation of an intercellular gap or an interendothelial gap or endocytosis. In some embodiments, the intercellular gap or the interendothelial gap ranges from about 10 nm to about 10 um. In some embodiments, the stable vibration cavitation of the sonoactive microstructures moves the nucleic acid construct from an intravenous space into an interstitial space or into the cytoplasm.

In some embodiments, applying the ultrasonic acoustic energy at the second MI induces inertial cavitation of the sonoactive microstructures to disrupt the sonoactive microstructures. In some embodiments, applying the ultrasonic acoustic energy at the second MI induces inertial cavitation of the sonoactive microstructures to disrupt the sonoactive microstructures in a vasculature space and an extravascular space. In some embodiments, the extravascular spaces comprise an interstitial space, a subcutaneous space, intramuscular or a lymphatic space. In some embodiments, the extravascular spaces comprise an extravascular tissue. In some embodiments, the extravascular tissue comprises an interstitial space, a cytoplasmic space, a subcutaneous, a lymph tissues, muscular or combinations thereof.

In some embodiments, applying the ultrasonic acoustic energy at the second MI induces formation of a pore in a membrane of the cell. In some embodiments, the formation of a pore in a membrane of the cell ranges from about 10 nm to about 10 um.

In some embodiments, the ultrasound acoustic energy is applied using an ultrasound probe applying ultrasound acoustic energy to the tissue. In some embodiments, the acoustic radiation force is applied using an ultrasound probe applying ultrasound acoustic energy to the tissue. In some embodiments, the ultrasound probe comprises a plurality of piezoelectric elements configured to emit ultrasound acoustic energy. In some embodiments, portions of the plurality of piezoelectric elements are arranged in one or more arrays. In some embodiments, the ultrasound probe is a phased array transducer comprising a plurality of piezoelectric elements configured to emit ultrasound acoustic energy. In some embodiments, the ultrasound probe is a phased array ultrasound probe, a linear ultrasound probe, a curvilinear ultrasound probe, a convex array ultrasound probe, an endocavitary ultrasound probe, a 3D ultrasound probe, a 4D ultrasound probe, a Doppler ultrasound probe, or a color doppler ultrasound probe.

In some embodiments, administration of the sonoactive microstructures and nucleic acid constructs occurs simultaneously in that the sonoactive microstructures are mixed with a solution comprising the nucleic acid constructs prior to delivery to the subject. Such mixtures can comprise of 50% v/v of the sonoactive microstructures (e.g., Optison) and 50% v/v of a solution comprising a nucleic acid construct. Such mixtures can comprise varying percentages 5-90% v/v of the sonoactive microstructures.

In some embodiments, the nucleic acid construct comprises a miniplasmid backbone. As used herein, the term “miniplasmid (mpDNA)” refers to nucleic acid constructs that are smaller in size (i.e., contain fewer base pairs (bp)) than conventional plasmids or pDNA. In some embodiments, mpDNA constructs comprise a backbone smaller than 1 kb. In some embodiments, mpDNA constructs are smaller than 1000 bp excluding an expression cassette. In some embodiments, mpDNA constructs comprise a backbone smaller than 0.5 kb. In some embodiments, mpDNA constructs are smaller than 500 bp excluding an expression cassette. In some embodiments, the miniplasmid does not comprise a bacterial origin of replication. As used herein, the term “Nanoplasmid™” (e.g., Nanoplasmid sourced from Aldevron, Fargo, South Dakota.) refers to a small mpDNA construct that has a plasmid backbone that is less than 500 bp and does not contain an antibiotic resistance gene.

Miniplasmid DNA nucleic acid constructs can be utilized to deliver an expression cassette, a transgene, or a nonendogenous gene to cells in target cell-types, tissues or organs. In some embodiments, the miniplasmid comprises less than 1000 base pairs excluding an expression cassette. In some embodiments, the miniplasmid comprises less than 500 base pairs excluding an expression cassette. In some embodiments, the miniplasmid does not comprise antibiotic resistant genes. In some embodiments, the miniplasmid does not comprise a bacterial genome. In some embodiments, the miniplasmid comprises a therapeutic transgene and/or a regulatory element. In some embodiments, the miniplasmid is a nanoplasmid. In some embodiments, the miniplasmid construct enhances the expression of the nonendogenous gene or a therapeutic transgene when used in conjunction with the claimed methods and ultrasound acoustic profiles. In some embodiments, the nanoplasmid construct enhances the expression of the nonendogenous gene or a therapeutic transgene. In some embodiments, durability of expression of a protein encoded by the nucleic acid payload may be increased relative to expression of the same protein in a larger plasmid (e.g., a plasmid of greater than 2 kb in length, excluding the transgene). In some embodiments, durability of expression of a protein encoded by the nucleic acid payload may be increased relative to expression of the same protein in another nucleic acid construct.

In some embodiments, the nucleic acid construct is a miniplasmid e.g., a construct comprising a backbone of less than 1000 bp or less than 500 bp) coupled to a nucleic acid payload.

In some embodiments, the nucleic acid payload comprises an expression cassette. In some embodiments, the expression cassette comprises a transgene. In some embodiments, the nucleic acid payload comprises a transgene (endogenous or non-endogenous). In some embodiments, the transgene comprises a therapeutic transgene. In some embodiments, inducing expression of the nucleic acid payload comprises inducing expression of the therapeutic transgene. In some embodiments, the transgene comprises a detectible marker. In some embodiments, the transgene comprises luciferase. In some embodiments, inducing expression of the nucleic acid payload comprises inducing expression of luciferase.

In some embodiments, a nucleic acid payload comprises a regulatory element such as a promoter, (e.g., APOE-ATT). In some embodiments, a total amount (e.g., dose) of DNA administered to a subject for purposes of sonoporation can range from 100 microgram to 200 mg.

In some embodiments, the therapeutic payload is a nonendogenous gene. In some embodiments, the nucleic acid payload is configured to perform gene augmentation, gene replacement, gene editing, gene knockdown, or gene knockout.

In some embodiments, the nucleic acid construct comprises one or more regulatory elements, such as a promoter, enhancer, ribosome binding site, or transcription termination signal. Examples of promoters contemplated herein include, but are not limited to, e.g., CMV promoter, UbC promoter, CAG promoter, EF-1α promoter, ApoE promoter, ApoE-AAT1 promoter, 3XSERP promoter, or P3-hybrid promoter. In some embodiments, the nucleic acid construct comprises a promoter sequence comprising CAG. In some embodiments, the nucleic acid construct comprises a promoter sequence comprising ApoE. In some embodiments, the nucleic acid construct comprises a promoter sequence comprising SERP. In some embodiments, the nucleic acid construct comprises a promoter sequence comprising P3.

In some embodiments, inducing expression of the nucleic acid payload comprises inducing production of RNA encoded by the payload. In some embodiments, inducing expression of the nucleic acid payload comprises inducing production of protein encoded by the payload.

In some embodiments, the payload comprises a therapeutic RNA. In some embodiments, the therapeutic RNA is an mRNA. In some embodiments, the therapeutic RNA is an RNA interference (RNAi) agent, e.g., a double-stranded RNA, a single-stranded RNA, a micro-RNA (miRNA), a short interfering RNA (siRNA), short hairpin RNA (shRNA), or a triplex-forming oligonucleotide. In some embodiments, the therapeutic RNA is a catalytically active RNA molecule (ribozyme). In some embodiments, the therapeutic RNA is a transfer RNA (tRNA). In some embodiments, the therapeutic RNA comprises one or more chemical modifications (e.g., one or more modified nucleobases, nucleosides, or nucleotides). In some embodiments, the nucleic acid construct is configured to perform gene augmentation, gene replacement, base editing, base knockdown, gene editing gene knockdown, or gene knockout. In some embodiments, delivering the nucleic acid payload to the target cell of the subject increases or decreases expression of a gene in the target cell.

In some embodiments, the payload comprises one or more components of a gene editing system. In some embodiments, the payload comprises a nuclease or engineered nuclease suitable for gene editing. In some embodiments, the nuclease is delivered as a polypeptide. In some embodiments, the nuclease is delivered as a nucleic acid encoding the nuclease. In some embodiments, the gene editing system is a CRISPR/Cas system. In some embodiments, the payload comprises a gRNA or a nucleic acid molecule encoding a gRNA (e.g., a plasmid encoding the gRNA). In some embodiments, the payload comprises a Cas protein or homologs or variants thereof, or a nucleic acid molecule encoding the Cas protein or homologs or variants thereof. In some embodiments, the payload comprises a TALEN or a nucleic acid molecule encoding the TALEN. In some embodiments, the payload comprises a zinc-finger nuclease (ZFN) or a nucleic acid encoding the ZFN. In some embodiments, the nuclease is an engineered nuclease. In some embodiments, the engineered nuclease is catalytically inactive. In some embodiments, the engineered nuclease is a fusion protein comprising the engineered nuclease a regulatory protein or an enzyme, or a functional domain thereof (e.g., a nuclease fused to a transcriptional regulatory domain or a nuclease fused to a deaminase) In some embodiments, the payload may further comprise a template DNA molecule suitable for knock-in to the subject's genome via non-homologous end joining (NHEJ) or homology directed repair (HDR).

Sonoactive microstructures (also referred to as acoustic microspheres or “microbubbles”) contemplated herein include, but are not limited to, those used as ultrasonic imaging contrast agents. In some embodiments, the sonoactive microstructures comprise a phospholipid stabilized microstructure. In some embodiments, the phospholipid stabilized microstructure comprises a high molecular wight gas core, or a perflutran core. Examples of sonoactive microstructures include, but are not limited to, OPTISON (GE Healthcare), Sonazoid (GE Healthcare), or DEFINITY and Definity RT (Lantheus Medical Imaging, Inc).

In some embodiments, the sonoactive microstructures are LUMASON (Bracco) (sulfur hexafluoride lipid-type A microspheres). In some embodiments, the sonoactive microstructures are SonoVue (sulfur hexafluoride microbubbles). In some embodiments, the sonoactive microstructures comprise a protein stabilized microstructure. In some embodiments, the sonoactive microstructures are Optison microbubbles.

The sonoactive microstructures can be administered prior to, after, or simultaneous (e.g., co-administered) with the administration of the nucleic acid construct (or nucleic acid payload). In some embodiments, the nucleic acid construct and the sonoactive microstructures are coadministered. In some embodiments, the administering of the nucleic acid construct and the sonoactive microstructures occurs serially, concurrently, sequentially, or continuously. In some embodiments, the administering of the nucleic acid construct and the sonoactive microstructures occurs serially. In some embodiments, the administering of the nucleic acid construct and the sonoactive microstructures occurs concurrently. In some embodiments, the administering of the nucleic acid construct and the sonoactive microstructures occur sequentially. In some embodiments, the administering of the nucleic acid construct and the sonoactive microstructures occurs continuously.

In some embodiments, the nucleic acid construct is administered at a dosage up to about 1.5 mg/kg bodyweight. In some embodiments, the nucleic acid construct is administered at a dosage up to about 0.2 mg/kg bodyweight. In some embodiments, the nucleic acid construct is administered at a dosage between about 0.4 mg/kg bodyweight to about 1.5 mg/kg bodyweight. In some embodiments, the linear dsDNA construct is administered at a first dosage that is less than 500 micrograms. In some embodiments, the linear dsDNA construct is administered to the subject at a concentration of about 3.5 μg/μl. In some embodiments, at least 2×10¹³ copies of the nucleic acid construct are administered to the subject. In some embodiments, about 2×10¹³ to about 3×10¹³ copies of the nucleic acid construct are administered to the subject. In some embodiments, each nucleic acid construct comprises a copy of a transgene.

In some embodiments, the sonoactive microstructures are administered at a dosage of about 1-50 mL, for example 1 mL of Optison. The sonoactive microstructures may be administered at a concentration of about 5 M to about 8 M microstructures per mL. In some embodiments, the sonoactive microstructures are administered at a concentration of about 5×10\hat{ }8 to about 1.2×10\hat{ }9 microstructures/mL, for example 1×10\hat{ }9 of Definity RT. In some embodiments, the sonoactive microstructures are administered at a concentration of about 0.1 to about 0.8 mg/kg. In some embodiments, the sonoactive microstructures are administered at a concentration of about 0.1 to about 1.0 mL/kg. In some embodiments, the sonoactive microstructures are administered at a concentration of about 10\hat{ }9 microstructures/mL. In some embodiments, the sonoactive microstructures are administered at a concentration of about 5×10\hat{ }8 to about 8×10\hat{ }8 microstructures/mL.

As used herein, concentrations of microstructures/mL refer to the concentration of the sonoactive microstructures in a pharmaceutical composition immediately prior to administration to the subject. In some embodiments, the sonoactive microstructures are administered at a concentration of about 5×10⁸ to about 1.2×10¹⁰ microstructures/mL. In some embodiments, the sonoactive microstructures are administered at a dosage of about 1-50 mL, for example 1 mL of a protein-stabilized sonoactive microstructure (e.g., Optison). In some embodiments, the protein-stabilized sonoactive microstructure (e.g., Optison) has a diameter of 3-4.5 micrometers. The sonoactive microstructures may be administered at a concentration of about 5 M (million) to about 8 M microstructures per mL. In some embodiments, 1×10⁹ of phospholipid stabilized sonoactive microstructures (e.g., Sonazoid) are administered. In some embodiments, the phospholipid stabilized sonoactive microstructures (e.g., Sonazoid) comprise a diameter of 1-5 micrometers. In some embodiments, the sonoactive microstructures are administered at a concentration of about 0.1 to about 0.8 mg/kg. In some embodiments, the sonoactive microstructures are administered at a concentration of about 0.1 to about 1.0 mL/kg. In some embodiments, the sonoactive microstructures are administered at a concentration of about 10\hat{ }9 microstructures/mL. In some embodiments, the sonoactive microstructures are administered at a concentration of at least 5×10\hat{ }8 microstructures per mL. In some embodiments, the sonoactive microstructures are administered at a concentration of up to 1.2×10\hat{ }10 microstructures/mL. In some embodiments, the sonoactive microstructures are administered at a concentration of 5×10\hat{ }8 to 8×10\hat{ }8 microstructures/mL.

In some embodiments, the nucleic acid construct and the sonoactive microstructures are mixed prior to being coadministered. In some instances, the sonoactive microstructures are mixed with the nucleic acid constructs before administering to the subject. In some instances, the sonoactive microstructures are mixed with the nucleic acid constructs along with additional buffers or agents such as saline or other biocompatible solutions with varying electrostatic charges and surface chemistries and ligands before administering to the subject. For example, Optison sonoactive microstructures can be mixed with a Nanoplasmid comprising a promoter operatively linked to a transgene (e.g., APOE-Fluc) and saline and are administered together. In some embodiments, administration of the sonoactive microstructures and nucleic acid constructs occurs simultaneously in that the sonoactive microstructures are mixed with a solution comprising the nucleic acid constructs prior to delivery to the subject. Such mixtures can comprise of 50% v/v of the sonoactive microstructures (e.g., Optison) and 50% v/v of a solution comprising a nucleic acid construct. Such mixtures can comprise varying percentages 5-90% v/v of the sonoactive microstructures.

In some embodiments, the administering of the nucleic acid construct and the sonoactive microstructures is by intravenous administration or subcutaneous or intramuscular or intra-arterial or inter-osseus or direct organ puncture. In some embodiments, administration of the sonoactive microstructures and nucleic acid constructs occurs simultaneously in that the sonoactive microstructures are mixed with a solution comprising the nucleic acid constructs prior to delivery to the subject. Such mixtures can comprise of 50% v/v of the sonoactive microstructures (e.g., Optison) and 50% v/v of a solution comprising a nucleic acid construct. Such mixtures can comprise varying percentages 5-90% v/v of the sonoactive microstructures. In some cases, mixtures may comprise a ratio of one part solution comprising the nucleic acid constructs to four or more parts solution comprising the sonoactive microstructures.

In some embodiments, after administering of the nucleic acid construct and sonoactive microstructures, the ultrasound acoustic energy is applied at the target cell, tissue, or organ.

Once the nucleic acid constructs are inside the target cell, expression of the nucleic acid payload is induced. In some embodiments, the nucleic acid payload comprises luciferase. In some embodiments, inducing expression of the nucleic acid payload using the miniplasmid construct comprises inducing expression inducing an average radiance of at least 2×10\hat{ }4 p/sec/cm2/sr. In some embodiments, inducing expression of the nucleic acid payload comprises inducing an average radiance of from about 2×10\hat{ }4 p/sec/cm2/sr to about 5×10\hat{ }5 p/sec/cm2/sr.

In some embodiments, inducing expression of the nucleic acid payload comprises inducing a flux of at least 10\hat{ }6 p/s. In some embodiments, inducing expression of the nucleic acid payload comprises inducing a flux of about 10\hat{ }6 p/s to about 10\hat{ }9 p/s.

In some embodiments, inducing expression of the nucleic acid payload comprises inducing a flux which is 2, 3, 4, or 5× greater than expression induced without repeating applying the ultrasonic acoustic energy at the first MI, and the applying the ultrasonic acoustic energy at the second MI.

In some embodiments, inducing expression of the nucleic acid payload comprises inducing expressing within about 3 to about 12 hours of administering the pay load. In some embodiments, inducing expression of the nucleic acid payload comprises inducing expression within about 3 hours of administration. In some embodiments, inducing expression of the nucleic acid payload comprises inducing expression within about 6 hours of administration. In some embodiments, inducing expression of the nucleic acid payload comprises inducing expression within about 12 hours of administration.

Undesirable effects on living cells or tissues can occur due to ultrasound applications. In some embodiments, the present disclosure provides methods for improvement of gene transfection and not result in substantial DNA or cell damage in the target cells, tissues, or organs, using sonoporation by alternating ultrasonic acoustic energy between the first MI and the second MI. In some embodiments, the method does not result in substantial cellular damage to the target cell. In some embodiments, the method results in less than 1%, 5%, or 10% of target cells undergoing apoptosis.

Cellular damage can be detected using apoptotic biomarkers. For example, in liver, detection of released hepatocellular transaminases, e.g., serum alanine aminotransferase (ALT) or aspartate aminotransferase (AST), can be an indicator of apoptotic hepatocytes. Additional apoptotic biomarkers comprise interleukin 6 (IL6) or B-cell lymphoma 2 (BCL2 or BCL2 apoptosis regulator). In some embodiments, the following biomarkers for cellular damage are not detected at apoptotic levels following delivering the nucleic acid payload to the target cell of the subject: ALT, AST, IL6, BCL2, or combinations thereof. In some embodiments, the following biomarkers for cellular damage are not clinically elevated following delivering the nucleic acid payload to the target cell of the subject: ALT, AST, IL6, BCL2, or combinations thereof. In some embodiments, the following biomarkers for cellular damage are not detected at apoptotic levels following delivering the nucleic acid payload to the target cell of the subject: ALT, AST, IL6, BCL2, or combinations thereof, and, optionally wherein the target cell is in a liver. In some embodiments, the following biomarkers for cellular damage are not clinically elevated following delivering the nucleic acid payload to the target cell of the subject: ALT, AST, IL6, BCL2, or combinations thereof, and, optionally wherein the target cell is in a liver. In some embodiments, the following biomarkers for cellular damage are not clinically elevated following delivering the nucleic acid payload to the target cell of the subject: creatinine levels in urine, albumin to creatine ratio in urine, creatinine levels in blood, a glomerular filtration rate, blood in urine, protein levels in urine, or an osmolality of urine, and, optionally wherein the target cell is in a kidney. In some embodiments, the following biomarkers for cellular damage are not clinically elevated following delivering the nucleic acid payload to the target cell of the subject: troponin levels in blood, or creatinine phospho kinase, and, optionally wherein the target cell is in a heart or skeletal muscle.

A sonoporation treatment using the methods described herein can be used to induce expression of a nucleic acid payload in a cell in a liver or a cell in a kidney.

A sonoporation treatment using the methods described herein can be used to treat a subject in need of gene therapy or enzyme replacement treatment. In another aspect, the present disclosure provides methods of treating a subject having a liver condition. In some embodiments, the liver condition treated is: Wilson's Disease, Cholestasis progressive familial intrahepatic, Von Willebrand disease, Hemophilia A, Hemophilia B, Factor 5 deficiency, Alpha-Mannosidosis, Gaucher's (glucocerebrosidase deficiency, glucocerebrosidosis), Niemann Pick Disease A/B, Carbamoylphosphate Synthetase I Deficiency, Glycogen Storage Disease Type III, Cystinosis, A1AT deficiency, Citrullinemia Type I & II.

In some embodiments, the present disclosure provides methods of treating a subject having a liver condition with a therapeutic transgene. In some embodiments, the therapeutic transgene encodes one or more of: ATP7B; ABCB11; ABCB4; ATP8B1; TJP2; VWF; FVIII; FIX; F5; MAN2B1; GBA; SMPD1; CPS1; GDE/AGL; CTNS; SERPINA1; ASS1, and/or SLC25A13.

In some embodiments, the present disclosure provides methods of treating a subject having a liver condition with a therapeutic transgene. In some embodiments, the liver condition is Wilson's Disease, and the therapeutic transgene encodes ATP7B. In some embodiments, the liver condition is Cholestasis, progressive familial intrahepatic (PFIC1-4) and the therapeutic transgene encodes one or more of ABCB11, ABCB4, ATP8B1 and/or TJP2. In some embodiments, the liver condition is Von Willebrand Disease and the therapeutic transgene encodes VWF. In some embodiments, the liver condition is Hemophilia A, and the therapeutic transgene encodes FVIII. In some embodiments, the liver condition is Hemophilia B, and the therapeutic transgene encodes FIX. In some embodiments, the liver condition is Factor V Deficiency, and the therapeutic transgene encodes F5. In some embodiments, the liver condition is Alpha-Mannosidosis, and the therapeutic transgene encodes MAN2B1. In some embodiments, the liver condition is Gaucher's (glucocerebrosidase deficiency, glucocerebrosidosis), and the therapeutic transgene encodes GBA . In some embodiments, the liver condition is Niemann Pick Disease A/B, and the therapeutic transgene encodes SMPD1. In some embodiments, the liver condition is Carbamoylphosphate Synthetase I Deficiency, and the therapeutic transgene encodes CPS1. In some embodiments, the liver condition is Glycogen Storage Disease Type III:, and the therapeutic transgene encodes GDE/AGL. In some embodiments, the liver condition is Cystinosis, and the therapeutic transgene encodes CTNS. In some embodiments, the liver condition is A1AT deficiency, and the therapeutic transgene encodes SERPINA. In some embodiments, the liver condition is Citrullinemia Type I & II, and the therapeutic transgene encodes one or more of ASS1 and/or SLC25A13. In some embodiments, the methods comprise (a) administering to the subject a nucleic acid construct comprising the nucleic acid payload (e.g., a therapeutic transgene); (b) administering to the subject a plurality of sonoactive microstructures; and (c) administering a sonoporation treatment. In some embodiments, the sonoporation treatment comprises applying an ultrasonic acoustic energy to a liver at a first mechanical index (MI) that is less than 0.4; (d) applying an ultrasonic acoustic energy to the liver at a second MI that is greater than 0.4 and less than 2.0. In some embodiments, the method comprises repeating applying the ultrasonic acoustic energy at the first MI, and the applying the ultrasonic acoustic energy at the second MI a number of times. In some embodiments, the method comprises delivering the nucleic acid payload and the plurality of sonoactive microstructures systemically (e.g., by intravenous administration).

In some embodiments, provided herein is a method of treating a subject having Hemophilia A comprising administering to the subject a nucleic acid construct comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0).

In some embodiments, provided herein is a method of treating a subject having Wilson's Disease comprising administering to the subject a nucleic acid construct comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0). In some embodiments, the therapeutic transgene comprises a nucleic acid sequence encoding ATP7B. In some embodiments, the nucleic acid construct and the plurality of sonoactive microstructures are administered systemically (e.g., by intravenous administration).

In one aspect, using the methods described herein, the present disclosure provides methods of treating a subject having a kidney condition. In some embodiments, the kidney condition treated is: Alport Syndrome, or Autosomal Dominant Polycystic Kidney Disease.

In some embodiments, the present disclosure provides methods of treating a subject having a kidney condition with a therapeutic transgene. In some embodiments, the therapeutic transgene encodes one or more of COL4A3, COL4A4, COL4A5, PKD1 and/or PKD2.

In some embodiments, the present disclosure provides methods of treating a subject having a kidney condition with a therapeutic transgene. In some embodiments, the kidney condition is Alport Syndrome, and the therapeutic transgene encodes one or more of COL4A3, COL4A4, and/or COL4A5. In some embodiments, the kidney condition is Autosomal Dominant Polycystic Kidney Disease, and the therapeutic transgene encodes one or more of PKD1 and/or PKD2. In some embodiments, the methods comprise (a) administering to the subject a nucleic acid construct comprising the nucleic acid payload; (b) administering to the subject a plurality of sonoactive microstructures; and (c) administering a sonoporation treatment. In some embodiments, the sonoporation treatment comprises applying an ultrasonic acoustic energy to a kidney at a first mechanical index (MI) that is less than 0.4; (d) applying an ultrasonic acoustic energy to the kidney at a second MI that is greater than 0.4 and less than 2.0.

In some embodiments, provided herein is a method of treating a subject having Alport Syndrome comprising administering to the subject a nucleic acid construct comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0). In some embodiments, the therapeutic transgene comprises a nucleic acid sequence encoding COL4A3. In some embodiments, the therapeutic transgene comprises a nucleic acid sequence encoding COL4A4. In some embodiments, the therapeutic transgene comprises a nucleic acid sequence encoding COL4A5. In some embodiments, the nucleic acid construct and the plurality of sonoactive microstructures are administered systemically (e.g., by intravenous administration).

In some embodiments, provided herein is a method of treating a subject having Autosomal Polycystic Kidney Disease comprising administering to the subject a nucleic acid construct comprising a therapeutic transgene; administering to the subject a plurality of sonoactive microstructures; applying an ultrasonic acoustic energy to the target cell at a first mechanical index (MI) that is up to 0.4 (e.g., 0<MI≤0.4); and applying an ultrasonic acoustic energy to the target cell at a second MI that is greater than 0.4 and up to 2.0 (e.g., 0.4<MI≤2.0). In some embodiments, the therapeutic transgene comprises a nucleic acid sequence encoding PKD1. In some embodiments, the therapeutic transgene comprises a nucleic acid sequence encoding PKD2. In some embodiments, the nucleic acid construct and the plurality of sonoactive microstructures are administered systemically (e.g., by intravenous administration).

In another aspect, the present disclosure provides a kit to perform the methods described herein. In some embodiments, the kit comprises: (a) a first container comprising microbubbles for sonoporation; and (b) a second container comprising miniplasmids comprising a transgene and a mixture chamber (reservoir, syringe, Y-port, etc.).

In some embodiments, the miniplasmid further comprises an expression cassette. As used herein, an expression cassette comprises nucleic acid sequences encoding nucleic acid payload, e.g., an expression cassette comprising a transgene. The expression cassette further comprises a regulatory element such as a promoter, enhancer, ribosome binding site, or transcription termination signal.

In some embodiments, the first container and second container are configured to induce the expression of the transgene in the target cell of the subject within 20 hours after the transfection.

In some embodiments, the method further includes inducing expression of the nucleic acid payload and maintaining expression of a protein encoded by the nucleic acid payload for at least 1, 2, 3, 4, 5, 6, or 7 days following administration of the nucleic acid construct, the sonoactive microstructures, and application of the ultrasonic acoustic energy to the target cell at the low MI and the high MI. In some embodiments, the method further includes inducing expression of the nucleic acid payload and maintaining expression of a protein encoded by the nucleic acid payload for at least 1, 2, 3, 4, 5, 6, or 7 days following administration of the nucleic acid construct, the sonoactive microstructures, and application of the ultrasonic acoustic energy to the target cell at the low MI and the high MI.

In some embodiments, the method further includes increasing expression of the nucleic acid payload by increasing the dosage of the nucleic acid payload administered to the subject. In some embodiments, the method further includes increasing expression of the nucleic acid payload by increasing the dosage of the nucleic acid payload administered to the subject in a linear manner. In some embodiments, the method further includes increasing expression of the nucleic acid payload by administering at least 5, 50, 250, or 500 ug of the nucleic acid payload to the subject.

In some embodiments, ALT is not detected at levels exceeding 60, 65, 70, 75, 80, 85,90, 95, 100, 125, 150, 175, or 200 U/L following delivering the nucleic acid payload to the target cell of the subject. In some embodiments, AST is not detected at levels exceeding 225, 250, 275, or 300 U/L following delivering the nucleic acid payload to the target cell of the subject. In some embodiments, IL6 is not detected at levels exceeding 2, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4, 4.2, 4.4, 4.6, 4.8, 5, 5.2, 5.4, 5.6, 5.8, or 6 pg/mL following delivering the nucleic acid payload to the target cell of the subject.

In some embodiments, the kit further comprises instructions for software and hardware directions for the safe and effective operation of an ultrasound machine sufficient to disrupt the sonoactive microstructures to generate the sonoporation processes which include but are not limited to the following: disrupting the microstructures, inducing inertial and stable cavitation, promoting endocytosis and inter-endothelial gap formation, microstreaming at cell surfaces, thereby increasing transfection of a nucleic acid payload to a cell. In some embodiments, the instructions described methods for improvement of gene transfection using sonoporation by applying alternating ultrasonic acoustic energy between a first MI then a second MI. In some embodiments, the kit further comprises instructions for administration of the first container and the second container.

The present disclosure provides ultrasound systems comprising computer systems that are programmed to implement methods of the disclosure. The ultrasound systems 201 may be operably connected to one or more ultrasound transducers 206 controlled by one or more computer processers 204 which may comprise one or more computer readably medium 205/203/202 which comprise instructions configured to cause the ultrasound systems to perform the methods of the present disclosure. The ultrasound systems 201 and/or the computer processers 204 may be in communication with the cloud or other remote server which enable the remote operation and control of the ultrasound systems 201 and performance of the methods disclosed herein. The computer system can be an electronic device of a user or a computer system that is remotely located with respect to the electronic device. The electronic device can be a mobile electronic device. The computer system includes a central processing unit (CPU, also “processor” and “computer processor” herein), which can be a single core or multi core processor, or a plurality of processors for parallel processing. The computer system also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user (e.g., hand-held device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, concentration of the analyte of interest. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit.

In some respects, the disclosed provides quality control methods or methods to assess a risk associated with a food, with a hospital, with a clinic, or any other location where the presence of a bacterium poses a certain risk to one or more subjects. In many instances, systems, platforms, software, networks, and methods described herein include a digital processing device, or use of the same. In further embodiments, the digital processing device includes one or more hardware central processing units (CPUs), i.e., processors that carry out the device's functions, such as the automated sequencing apparatus disclosed herein or a computer system used in the analyses of a plurality of nucleic acid sequencing reads from samples derived from a food processing facility or from any other facility, such as a hospital a clinical or another. In further embodiments, the digital processing device further comprises an operating system configured to perform executable instructions. In some embodiments, the digital processing device is optionally connected a computer network. In further embodiments, the digital processing device is optionally connected to the Internet such that it accesses the World Wide Web. In further embodiments, the digital processing device is optionally connected to a cloud computing infrastructure. In other embodiments, the digital processing device is optionally connected to an intranet. In other embodiments, the digital processing device is optionally connected to a data storage device. In other embodiments, the digital processing device could be deployed on premise or remotely deployed in the cloud.

In accordance with the description herein, suitable digital processing devices include, by way of non-limiting examples, server computers, desktop computers, laptop computers, notebook computers, sub-notebook computers, netbook computers, netpad computers, set-top computers, handheld computers, Internet appliances, mobile smartphones, tablet computers, personal digital assistants, video game consoles, and vehicles. Those of skill in the art will recognize that many smartphones are suitable for use in the system described herein. Those of skill in the art will also recognize that select televisions, video players, and digital music players with optional computer network connectivity are suitable for use in the system described herein. Suitable tablet computers include those with booklet, slate, and convertible configurations, known to those of skill in the art. In many aspects, the disclosure contemplates any suitable digital processing device that can either be deployed to a food processing facility, or is used within said food processing facility to process and analyze a variety of nucleic acids from a variety of samples.

In some embodiments, a digital processing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smart phone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®.

In some embodiments, a digital processing device includes a storage and/or memory device. The storage and/or memory device is one or more physical apparatuses used to store data or programs on a temporary or permanent basis. In some embodiments, the device is volatile memory and requires power to maintain stored information. In some embodiments, the device is non-volatile memory and retains stored information when the digital processing device is not powered. In further embodiments, the non-volatile memory comprises flash memory. In some embodiments, the non-volatile memory comprises dynamic random-access memory (DRAM). In some embodiments, the non-volatile memory comprises ferroelectric random-access memory (FRAM). In some embodiments, the non-volatile memory comprises phase-change random access memory (PRAM). In other embodiments, the device is a storage device including, by way of non-limiting examples, CD-ROMs, DVDs, flash memory devices, magnetic disk drives, magnetic tapes drives, optical disk drives, and cloud computing-based storage. In further embodiments, the storage and/or memory device is a combination of devices such as those disclosed herein.

In some embodiments, a digital processing device includes a display to send visual information to a user. In some embodiments, the display is a cathode ray tube (CRT). In some embodiments, the display is a liquid crystal display (LCD). In further embodiments, the display is a thin film transistor liquid crystal display (TFT-LCD). In some embodiments, the display is an organic light emitting diode (OLED) display. In various further embodiments, on OLED display is a passive-matrix OLED (PMOLED) or active-matrix OLED (AMOLED) display. In some embodiments, the display is a plasma display. In other embodiments, the display is a video projector. In still further embodiments, the display is a combination of devices such as those disclosed herein.

In some embodiments, a digital processing device includes an input device to receive information from a user. In some embodiments, the input device is a keyboard. In some embodiments, the input device is a pointing device including, by way of non-limiting examples, a mouse, trackball, track pad, joystick, game controller, or stylus. In some embodiments, the input device is a touch screen or a multi-touch screen. In other embodiments, the input device is a microphone to capture voice or other sound input. In other embodiments, the input device is a video camera to capture motion or visual input. In still further embodiments, the input device is a combination of devices such as those disclosed herein.

In some embodiments, a digital processing device includes a digital camera. In some embodiments, a digital camera captures digital images. In some embodiments, the digital camera is an autofocus camera. In some embodiments, a digital camera is a charge-coupled device (CCD) camera. In further embodiments, a digital camera is a CCD video camera. In other embodiments, a digital camera is a complementary metal-oxide-semiconductor (CMOS) camera. In some embodiments, a digital camera captures still images. In other embodiments, a digital camera captures video images. In various embodiments, suitable digital cameras include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, and higher megapixel cameras, including increments therein. In some embodiments, a digital camera is a standard definition camera. In other embodiments, a digital camera is an HD video camera. In further embodiments, an HD video camera captures images with at least about 1280×about 720 pixels or at least about 1920×about 1080 pixels. In some embodiments, a digital camera captures color digital images. In other embodiments, a digital camera captures grayscale digital images. In various embodiments, digital images are stored in any suitable digital image format. Suitable digital image formats include, by way of non-limiting examples, Joint Photographic Experts Group (JPEG), JPEG 2000, Exchangeable image file format (Exif), Tagged Image File Format (TIFF), RAW, Portable Network Graphics (PNG), Graphics Interchange Format (GIF), Windows® bitmap (BMP), portable pixmap (PPM), portable graymap (PGM), portable bitmap file format (PBM), and WebP. In various embodiments, digital images are stored in any suitable digital video format. Suitable digital video formats include, by way of non-limiting examples, AVI, MPEG, Apple® QuickTime®, MP4, AVCHD®, Windows Media®, DivX™, Flash Video, Ogg Theora, WebM, and RealMedia.

In many aspects, the systems, platforms, software, networks, and methods disclosed herein include one or more non-transitory computer readable storage media encoded with a program including instructions executable by the operating system of an optionally networked digital processing device. For instance, in some aspects, the methods comprise creating data files associated with a plurality of sequencing reads from a plurality of samples associated with a food processing facility. In further embodiments, a computer readable storage medium is a tangible component of a digital processing device. In further embodiments, a computer readable storage medium is optionally removable from a digital processing device. In some embodiments, a computer readable storage medium includes, by way of non-limiting examples, CD-ROMs, DVDs, flash memory devices, solid state memory, magnetic disk drives, magnetic tape drives, optical disk drives, cloud computing systems and services, and the like. In some cases, the program and instructions are permanently, substantially permanently, semi-permanently, or non-transitorily encoded on the media.

In some embodiments, the systems, platforms, software, networks, and methods disclosed herein include at least one computer program. A computer program includes a sequence of instructions, executable in the digital processing device's CPU, written to perform a specified task. In light of the disclosure provided herein, those of skill in the art will recognize that a computer program may be written in various versions of various languages. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®. NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. A web application for providing a career development network for artists that allows artists to upload information and media files, in some embodiments, includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

In some embodiments, a computer program includes a mobile application provided to a mobile digital processing device. In some embodiments, the mobile application is provided to a mobile digital processing device at the time it is manufactured. In other embodiments, the mobile application is provided to a mobile digital processing device via the computer network described herein.

In view of the disclosure provided herein, a mobile application is created by techniques known to those of skill in the art using hardware, languages, and development environments known to the art. Those with skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite,. NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Android™ Market, BlackBerry® App World, App Store for Palm devices, App Catalog for webOS, Windows® Marketplace for Mobile, Ovi Store for Nokia® devices, Samsung® Apps, and Nintendo® DSi Shop.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB. NET, or combinations thereof. Compilation is often performed, at least in part, to create an executable program. In some embodiments, a computer program includes one or more executable complied applications.

In some embodiments, after a first body of the 3D object is produced, the movable stage is removed from the actuator system. The 3D object may then continue to further processing steps, such as a perfusion sequence as described herein. The systems, platforms, software, networks, and methods disclosed herein include, in various embodiments, software, server, and database modules. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### DEFINITIONS

As used in the specification and claims, the singular forms “a”, “an” and “the” include plural references unless the context clearly dictates otherwise. For example, the term “a sample” includes a plurality of samples, including mixtures thereof.

As used herein, the term “about” a number refers to that number plus or minus 10% of that number. The term “about” a range refers to that range minus 10% of its lowest value and plus 10% of its greatest value.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

### EXAMPLES

The following examples are provided to further illustrate some embodiments of the present disclosure but are not intended to limit the scope of the disclosure; it will be understood by their exemplary nature that other procedures, methodologies, or techniques known to those skilled in the art may alternatively be used.

### Example 1: Durable Transgene Expression Using Linear Double Stranded Dna Constructs

### Experimental Animals and Protocol

There were five experimental groups, each of which included 4 BALB/c mice, with the exception of a control group which contained 3 BALB/c mice. Three experimental groups were administered circular, double stranded miniplasmid DNA lacking a bacterial origin of replication and antibiotic resistance genes, and were administered 250, 500, or 1000 ug of DNA payload and ultrasound in a sonoporation treatment. The fourth control group was administered 1000 ug of DNA payload of circular, double stranded miniplasmid DNA lacking a bacterial origin of replication and antibiotic resistance genes, but were not administered any ultrasound. A fifth experimental group was administered 178.6 ug of linear closed end double stranded DNA payload and ultrasound in a sonoporation treatment.

Mice in this experiment each received one dose of a DNA construct and sonoactive microstructure mixture. For all groups except a control group, following the delivery of the DNA and sonoactive microstructure mixture, ultrasound (US) energy was delivered transcutaneously in the area proximal to the target organ, the liver. The control group did not receive US energy. For all groups, the DNA construct used for transfection comprised a luciferase gene. To gauge the expression of the luciferase payload, IVIS fluorescence imaging sessions of all mice were performed at multiple time points after the DNA construct and sonoactive microstructure mixture was delivered. An illustration of these experimental protocols is shown in FIG. 1B.

A dose of sonoactive microstructure and DNA solution is prepared by preparing the sonoactive microstructures as instructed on the label, remove from 4C storage and roll between the palms for 20 seconds; removing protective plastic and aluminum covering from Optison vial; placing 25 G needle through the rubber gasket to provide a pressure vent; and using 1.5 inch 18G needle to draw up 225 uL of Optison into a syringe (dead space of the needle included in the calculations). With the same needle and syringe, 75 uL of DNA payload is drawn into the syringe to combine the DNA and Optison. It is mixed in the syringe by rolling between the fingers until the solution is homogenous. The DNA +Optison solution is drawn out of the needle dead space. Then the 18 G needle is exchanged for a 25 G blunt needle for injection into a patent JVC catheter. Sonazoid

Each dose included three 100 μL doses of DNA payload and sonoactive microstructure mixture in PBS, each delivered in through the jugular vein catheter. The mpDNA and linear closed end dsDNA constructs comprised nucleic acids encoding a luciferase gene coupled to an ApoE promoter. Sonoactive microstructures, Optison™ microbubbles Sonazoid were delivered to the mice at a ratio of 1:1 (DNA solution: sonoactive microstructure solution), by volume.

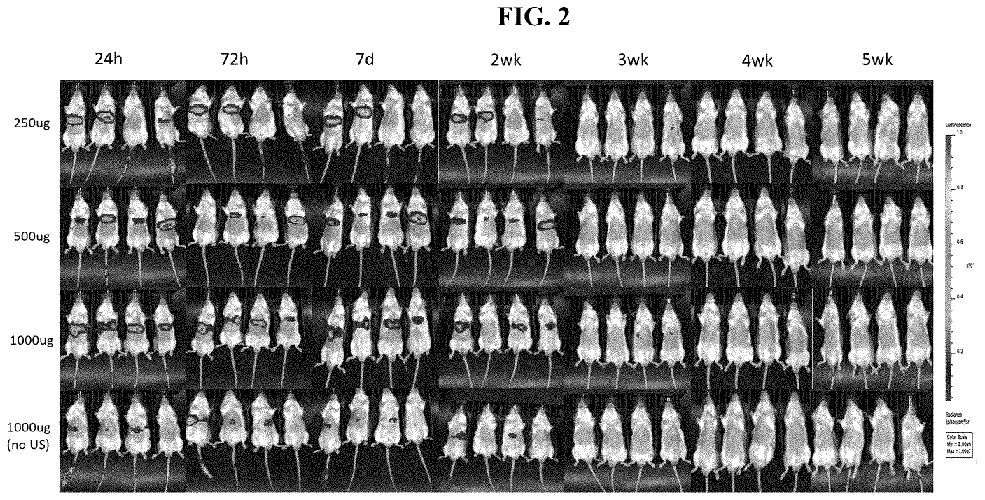

Four groups of mice received a dose of miniplasmid DNA (mpDNA) construct and sonoactive microstructures mixture in which the dose comprised either (1) 250 μg of mpDNA DNA construct with ultrasound (N=4 mice), (2) 500 μg of mpDNA DNA construct with ultrasound (N=4 mice), (3) 1000 μg of mpDNA DNA construct with ultrasound (N=4 mice), or (4) 1000 μg of mpDNA DNA construct without ultrasound (control group, N=3 mice) (FIG. 2 top row to bottom row, respectively). The mpDNA construct comprised a luciferase genetic payload coupled to an ApoE promoter. The fifth group of mice received (5) 178.6 μg of linear double stranded DNA payload and administration of ultrasound energy. The sonoactive agent used was Optison™ microbubbles. The mpDNA and microbubble mixture was delivered to mice at a ratio of 1:1 (DNA solution: sonoactive microstructures solution), by volume. The DNA and sonoactive microstructures mixture was delivered through the tail vein of the mice while the mice were conscious. The mpDNA construct utilized in these experiments was a Nanoplasmid™ DNA (Aldevron, SD) construct lacking a bacterial origin of replication and antibiotic resistance genes. The linear double stranded DNA construct utilized in this experiment was dbDNA (Toughlight Genetics Ltd., UK).

Following administration of the microbubbles and nucleic acid payload, ultrasound acoustic energy was delivered to the liver area of mice in these experiments using a L6-24 probe positioned perpendicular to the mouse to locate the lateral view of liver using B-mode ultrasound imaging at the low mechanical index (MI) value of 0.07. The depth setting was set to 2 cm, and a zoom of 0. Ultrasound was delivered continuously, and alternated between low mechanical index (MI) value of 0.07 and a high MI value of 0.8, without ceasing application of the ultrasound energy at any point during the treatment session. Nine flashes of high MI ultrasound at 0.8 were delivered with an interval of 4 seconds between each flash, and the administration of the 9 pulses was repeated three times. The high MI pulse duration was about 0.82 microseconds. The administration of the ultrasound was less than 30 seconds. The control group, group 4, received a mpDNA and microbubble mixture dose that comprised 1000 μg of mpDNA DNA construct but did not receive US energy.

One group of mice received a dose of linear double stranded DNA (dsDNA) construct and sonoactive microstructures mixture in which the dose comprised 178.6μg of doggy bone DNA (linear closed end dsDNA FIG. 1A) construct with ultrasound (N=4 mice). The linear dsDNA construct comprised a luciferase genetic payload coupled to a CAG promoter. The sonoactive agent used as Optison™ microbubbles. The linear closed end dsDNA and microbubble mixture was delivered to mice at a ratio of 1:1 (linear dsDNA solution: sonoactive microstructures solution), by volume. Following delivery of the linear closed end dsDNA and microbubble mixture, US energy was delivered continuously, and alternated between low mechanical index (MI) values and high MI values, without ceasing application of the ultrasound energy at any point during the treatment session. Nine flashes of high MI ultrasound were delivered with an interval of 8 seconds between each flash.

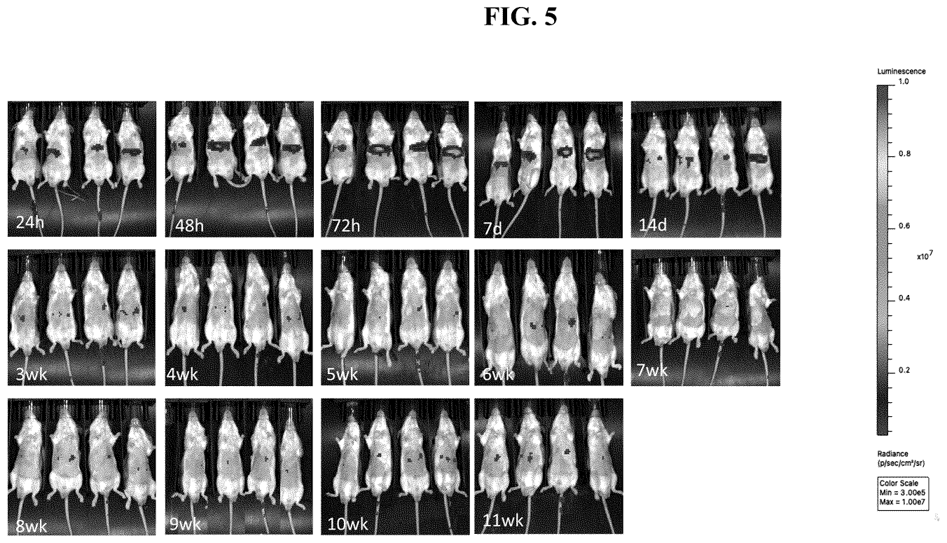

Using IVIS fluorescence imaging, all three groups of mice were imaged at 24 h, 72 h, 1, 2, 3, 4, 6, 7, 8, or 9, 10, 11, 12, 13, and 14 weeks after the dose of mpDNA and sonoactive microstructures mixture was delivered (FIG. 5; see also FIG. 6C). The linear closed end dsDNA and sonoactive microstructures mixture was delivered through the tail vein of the mice while the mice were conscious.

Results

Using IVIS, mice in groups that received the mpDNA and microbubble mixtures were imaged at 24 h, 72 h, 1, 2, 3, 4, 6, 7, 8, or 9, 10, 11, 12, 13, and 14 weeks after the dose of mpDNA and sonoactive microstructures mixture was delivered (FIG. 2). Mice in the control group to which no ultrasound was delivered (mpDNA group 4:1000 μg of mpDNA DNA construct without ultrasound), had, on average, a weaker fluorescence signal at each measurement than did mice in groups to which ultrasound was delivered (FIG. 4B, compare right most column to other columns in group; FIG. 4C, compare line with inverted triangle marker to other lines), of about 10'p/s/cm2/sr consistent with a background signal generated by the IVIS fluorescence radiance imaging, indicating in the control mice the lack of gene transfection and expression as result of the sonoporation treatments.

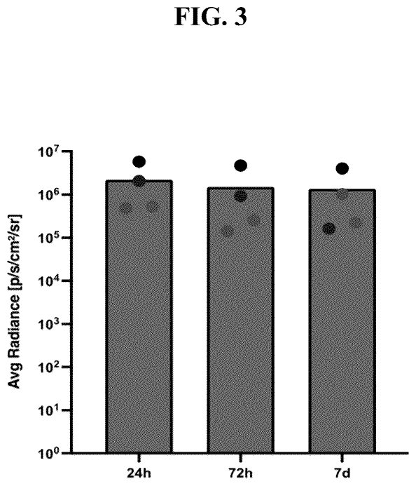

As measured by IVIS, the average fluorescence radiance in mice that received mpDNA and US energy was stable at the 24 h, 72 h, and 1 wk time points (FIG. 3 and FIG. 4A), indicating initial stable expression of the luciferase payload. However, the mean fluorescence radiance of these groups began dropping approximately two weeks after transduction (FIG. 4A-4C). Very little fluorescence was detected in mice after approximately three weeks from mpDNA delivery (FIG. 4A-4C), indicating a lack of stable gene expression at this point. Dots in the bar plots (FIG. 3, FIG. 4A and FIG. 4B) represent fluorescence values measured from individual mice, while the height of the bars represents the mean fluorescence value of the group at the time of measurement. Error bars in FIG. 3 and FIG. 4A-4C represent standard deviation.

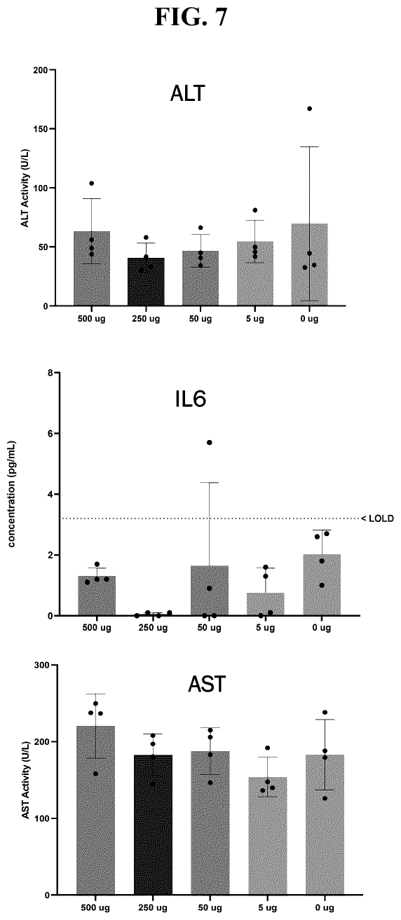

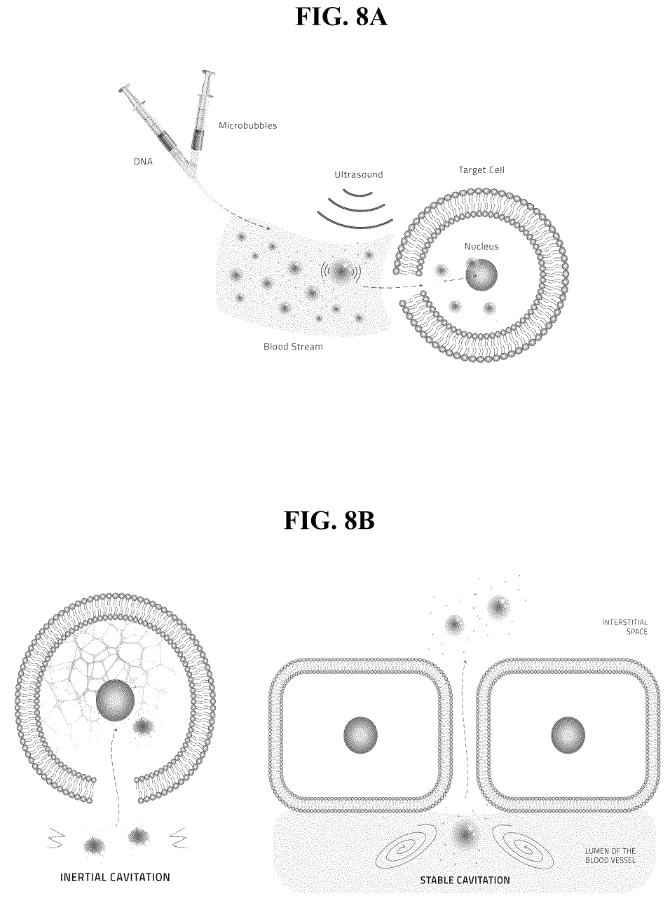

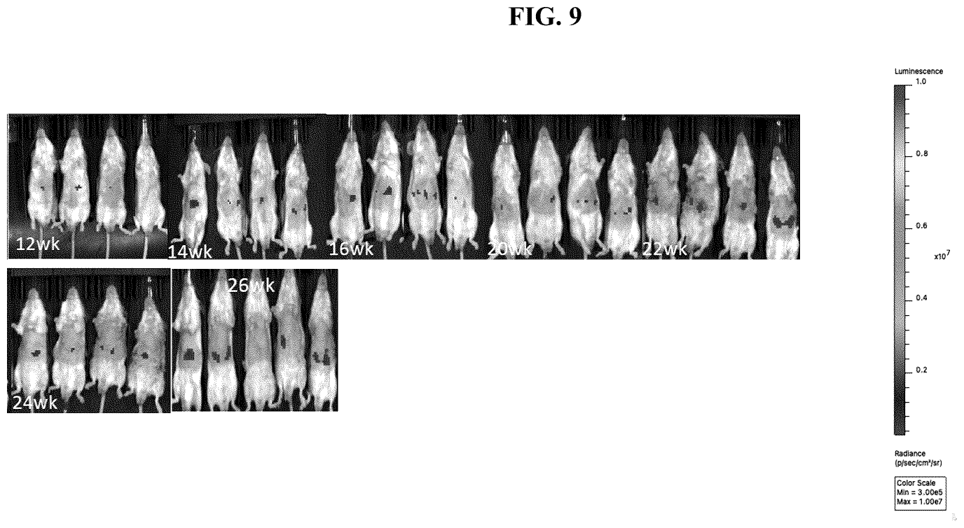

24 hours following administration of the first sonoporation treatment, serum ALT, IL6 and AST levels were measured to assess systemic inflammation, cellular inflammation, and cellular damage to the liver resulting from the sonoporation treatment. As is shown in FIG. 7, No change in liver enzymes 24 hours after delivery was observed, indicating a favorable response to the sonoporation treatment which did not result in significant cellular damage to the liver, or other cellular inflammatory response.

Mice in the group that received the linear closed end dsDNA and microbubble mixture were imaged at 24 h, 48 h, 72 h, 1, 2, 3, 4, 5, 6, 7, 8, 9, 10, and 11weeks after the dose of linear closed end dsDNA and sonoactive microstructures mixture was delivered (FIG. 5).

As measured by IVIS, the average fluorescence radiance in mice that received linear closed end dsDNA and US energy increased over the 24 h, 48 h, 72 h, and 1 wk measurements, and remained stable at about 10≡p/s/cm2/sr through 5 weeks (FIG. 6A and FIG. 6B), indicating an elevation in the expression of the luciferase payload in the week following administration sonoporation gene therapy treatment, and durable gene expression at the 5 week time point. In contrast the mean fluorescence radiance of the experimental groups administered the mpDNA construct began dropping approximately two weeks after transduction (FIGS. 6B and 6C), before ceasing to provide an IVIS signal greater than a background noise at about week 3. In contrast, detectable levels of fluorescence at an elevated level, and at the observed steady state, were seen at least fourteen weeks after transduction of the linear closed end dsDNA (FIG. 6C) , indicating the ability of linear closed end dsDNA transduction with US energy to induce long duration expression of a genetic payload. Dots in the bar plots (FIG. 6A, and FIG. 6C) represent fluorescence values measured from individual mice, while the height of the bars represents the mean fluorescence value of the group at the time of measurement. Error bars in FIG. 6A-6C represent standard deviation. Compared to the sonoporation treatments in which subjects were administered miniplasmid DNA failed to express the luciferase reporter gene after about 3 weeks, it is observed that the subjects administered linear double stranded DNA exhibited sustained and durable expression which reached a steady state at about 3 weeks, and which continued for at least 14 weeks.

### Example 2: Durable Transgene Expression Using Linear Double Stranded Dna Constructs

Experimental animals and protocol

Experimental group five from Example 1 continued observation for ongoing gene expression through 26 weeks to assess ongoing durability of gene expression.

In brief, the experimental group of mice received a dose of linear double stranded DNA (dsDNA) construct and sonoactive microstructures mixture in which the dose comprised 178.6 μg of dog bone DNA (linear closed end dsDNA, FIG. 1A) construct with ultrasound (N=4 mice). The linear dsDNA construct comprised a luciferase genetic payload coupled to a CAG promoter. The sonoactive agent used was Optison™ microbubbles. The linear closed end dsDNA and microbubble mixture was delivered to mice at a ratio of 1:1 (linear dsDNA solution: sonoactive microstructures solution), by volume. Following delivery of the linear closed end dsDNA and microbubble mixture, US energy was delivered continuously, and alternated between a low mechanical index (MI) value of 0.07 and a high MI value of 0.8, without ceasing contact with the skin of the subject and receiving or reflecting of the ultrasound energy at any point during the treatment session Nine flashes of high MI at 0.8 ultrasound were delivered with an interval of 8 seconds between each flash.

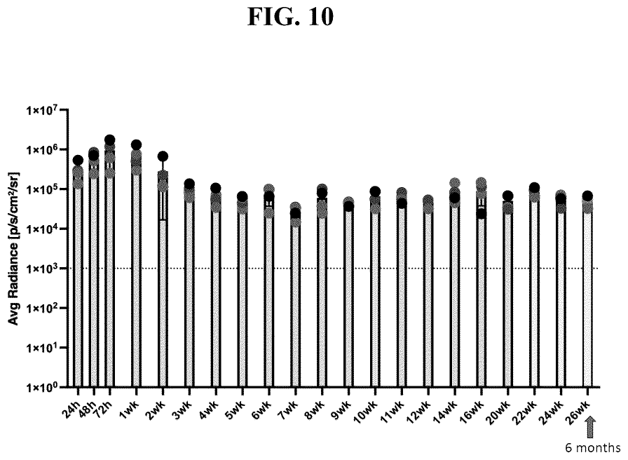

Using IVIS fluorescence imaging, all three groups of mice were imaged at 24 h, 48 h, 72 h, 1, 2, 3, 4, 5, 6, 7, 8, 9, 10, 11, 12, 14, 16, 20, 22, 24, or 26 weeks after the dose of linear closed end dsDNA and sonoactive microstructures mixture was delivered (FIG. 5, FIG. 6C, FIG. 9, and FIG. 10). The linear closed end dsDNA and sonoactive microstructures mixture were delivered through the tail vein of the mice while the mice were conscious.

### Results

Mice in the group that received the linear closed end dsDNA and microbubble mixture were imaged at 24 h, 48 h, 72 h, 1, 2, 3, 4, 5, 6, 7, 8, 9, 10, 11, 12, 14, 16, 20, 22, 24, or 26 weeks after the dose of linear closed end dsDNA and sonoactive microstructures mixture was delivered (FIG. 10).

Detectable levels of fluorescence at an elevated level, and at the observed steady state, were seen at least 26 weeks after transduction of the linear closed end dsDNA (FIG. 9 and FIG. 10), indicating the ability of linear closed end dsDNA transduction with US energy to induce long duration expression of a genetic payload. Compared to the sonoporation treatments in which subjects were administered miniplasmid DNA failed to express the luciferase reporter gene after about 3 weeks in Example 1, it is observed that the subjects administered linear double stranded DNA exhibited sustained and durable expression which reached a steady state at about 3 weeks, and which continued for at least 26 weeks.

### Example 3: Durable Transgene Expression Using Linear Double Stranded DNA Constructs

Experimental animals and protocol

In this example, gene expression from two experimental groups were evaluated, the first experimental group of mice received a 100 ug dose of a double stranded circular plasmid pDNA construct sonoactive agent mixture, and the second experimental group received 100 ug dose of a linear closed end double stranded DNA (dsDNA) construct and sonoactive agent mixture. Each experimental group included 3 animals of Rag2 mice, each of which was implanted with a jugular vein catheter. The linear dsDNA construct comprised a luciferase genetic payload coupled to an APOE-AAT promoter. The sonoactive agent used was Optison™ microbubbles. The linear closed end dsDNA and microbubble mixture was delivered to mice at a ratio of 1:1 (linear dsDNA solution: sonoactive microstructures solution), by volume. The plasmid pDNA construct was UC57 plasmid construct comprising the FLuc payload coupled to an APOE-AAT promoter. The linear closed end double stranded DNA construct utilized in this experiment was a double stranded linear close DNA made of FLuc payload coupled to an APOE-AAT promoter which is close-ended by non-coding sequences.

A dose of sonoactive microstructure and DNA solution is prepared by preparing the sonoactive microstructures as instructed on the label, remove from 4 C storage and roll between the palms for 20 seconds; removing protective plastic and aluminum covering from Optison vial; placing 25 G needle through the rubber gasket to provide a pressure vent; and using 1.5 inch 18 G needle to draw up 225 uL of Optison into a syringe (dead space of the needle included in the calculations). With the same needle and syringe, 250 uL of DNA payload in phosphate buffered saline is drawn into the syringe to combine the DNA and Optison. It is mixed in the syringe by rolling between the fingers until the solution is homogenous. The DNA +Optison solution is drawn out of the needle dead space. Then the 18 G needle is exchanged for a 25 G blunt needle for injection into a patent JVC catheter.

Mice were anesthetized with 2-3% isoflurane gas in an induction box prior to initiation of the experiment. Following delivery of the linear closed end dsDNA and microbubble mixture through the jugular vein catheter, US energy was delivered continuously, and alternated between a low mechanical index (MI) value of 0.07 and a high MI value of 1.5, without ceasing contact with the skin of the subject and receiving or transmitting of the ultrasound energy at any point during the treatment session Nine flashes of high MI at 1.5 ultrasound were delivered with an interval of 4 seconds between each flash. The focal depth was set to 2 cm and the gain was set to 28. Ultrasound acoustic energy was delivered to the liver area of mice in these experiments using a L6-24 probe positioned perpendicular to the mouse to locate the lateral view of liver using B-mode ultrasound imaging.

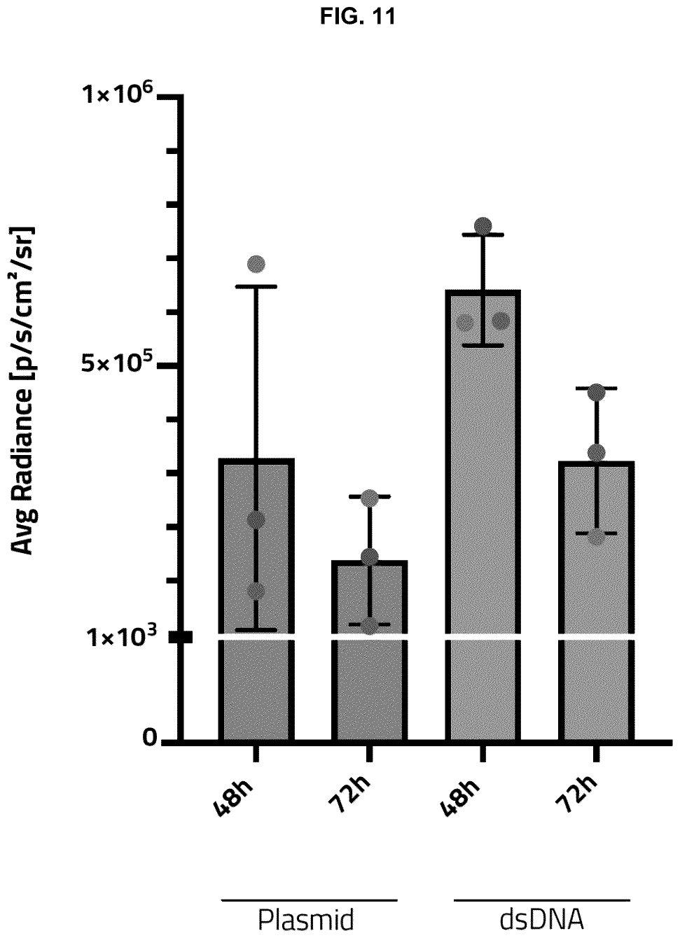

Following the sonoporation treatment session, gene expression was assessed using IVIS fluorescence imaging, all experimental groups of mice were imaged at 48 h and 72 h after the dose of linear closed end dsDNA and sonoactive microstructures mixture was delivered (FIG. 11).

### Results

Mice were imaged were imaged using IVIS to detect fluorescence produced by expression of the FLuc payload at 48 h and 72 h after the dose of DNA payload and the sonoactive agent were delivered in the sonoporation treatment (FIG. 11). Mice administered a plasmid DNA construct exhibited average radiance of about 3*10\hat{ }4 p/s/cm2/sr units at 48 hours, and average radiance of about 5*10\hat{ }3 p/s/cm2/sr units at 72 hours. Mice administered the linear closed end double stranded DNA construct exhibited increased average radiance of about 3*10\hat{ }5 p/s/cm2/sr units at 48 hours, and average radiance of about 3*10\hat{ }4 p/s/cm2/sr units at 72 hours. When compared to the mice administered a plasmid DNA construct, the mice administered the linear closed end double stranded DNA construct exhibited average radiance which was about one order of magnitude (e.g., 10-fold) higher, indicating that the linear closed end double stranded DNA construct provided significantly improved gene expression when compared to the pDNA construct administered under the same dosage and ultrasound conductions.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

What is claimed is:

1. A method of inducing an expression of a nucleic acid payload in target cell(s) of a mammalian subject comprising:
a. administering to the subject a linear double stranded DNA (dsDNA) construct covalently closed at each of its ends by DNA loops, the linear dsDNA construct comprising the nucleic acid payload;
b. administering to the subject a plurality of microbubbles; and
c. administering to the subject, in proximity to the target cell(s) an effective amount of an ultrasound energy, thereby inducing formation of pores the target cell(s) by disrupting the plurality of microbubbles to result in introduction of the dsDNA into the target cell(s) and expression of the nucleic acid payload in the target cell(s).

2. The method of any one of the preceding claims, wherein the linear dsDNA construct comprising the nucleic acid payload is administered at a first dosage that is less than, by mass, a dosage of DNA construct of a plasmid (pDNA) construct comprising the nucleic acid payload required to induce an equivalent level of payload expression in the subject when administered the effective amount of the ultrasound energy.

3. The method of any one of the preceding claims, wherein the linear dsDNA construct is administered to the subject at a dosage of up to about 0.2 mg/kg bodyweight.

4. The method of claim 2, wherein the first dosage of the dsDNA construct is at least 10%, 20%, 30% 40%, 50%, 60% or 70%, less than a second dosage of the plasmid (pDNA) construct.

5. The method of any one of the preceding claims, wherein the linear dsDNA construct is administered at a first dosage that is less than 500 micrograms.

6. The method of any one of the preceding claims, wherein the linear dsDNA construct is administered at a first dosage that is less than 1.5 mg/kg bodyweight.

7. The method of any one of the preceding claims, wherein the combination of (1) administering to the subject the dsDNA construct covalently closed at each of its ends by DNA loops; (2) administering to the subject the plurality of microbubbles; and (3) administering to the subject the effective amount of an ultrasound energy, thereby inducing expression of the nucleic acid payload in the target cell(s) induces expression of the nucleic acid payload within 24 hours.

8. The method of any one of the preceding claims, wherein the expression of a protein encoded by the nucleic acid payload in the target cell(s) is maintained for at least 1, 2, 3, 4, 5, 6, 7, 8, 9, 10, 11, 12, 13, 14, 15, 16, 17, 18, 19, 20, 21, 22, 23, 24, 25, or 26 weeks following performance of a.-c.

9. The method of any one of the preceding claims, wherein the expression of a protein encoded by the nucleic acid payload in the target cell(s) is maintained for at least 1, 2, 3, 4, 5, 6, 7, 8, 9, 10, 11, 12, 13, 14, 15, 16, 17, 18, 19, 20, 21, 22, 23, 24, 25, or 26 weeks following a single performance of a.-c.

10. The method of any one of the preceding claims, wherein the expression of a protein encoded by the nucleic acid payload in the target cell(s) is maintained at an elevated level as compared to a baseline expression level for at least 1, 2, 3, 4, 5, 6, 7, 8, 9, 10, 11, 12, 13, 14, 15, 16, 17, 18, 19, 20, 21, 22, 23, 24, 25, or 26weeks following the combination of (1) administering to the subject the dsDNA construct covalently closed at each of its ends by DNA loops; (2) administering to the subject the plurality of microbubbles; and (3) administering to the subject the effective amount of an ultrasound energy.

11. The method of any one of the preceding claims, wherein the expression of a protein encoded by the nucleic acid payload in the target cell(s) is maintained at a steady state at an elevated level as compared to a baseline expression level starting at least 3 weeks following the combination of (1) administering to the subject the dsDNA construct covalently closed at each of its ends by DNA loops; (2) administering to the subject the plurality of microbubbles; and (3) administering to the subject the effective amount of an ultrasound energy.

12. The method of any one of the preceding claims, wherein the expression of a protein encoded by the nucleic acid payload in the target cell(s) is maintained at a steady state at an elevated level as compared to a baseline expression level starting at least 3 weeks following performance of a.-c., and continuing to hold at the steady state thereafter for at least 1, 2, 3, 4, 5, 6, 7, 8, 9, 10, 11, 12, 13, 14, 15, 16, 17, 18, 19, 20, 21, 22, 23, 24, 25, or 26 weeks.

13. The method of any one of the preceding claims, wherein the steady state at the elevated level is within 1 order of magnitude of a peak level of expression.

14. The method of any one of the preceding claims, wherein the administering the effective amount of ultrasound energy comprises continuously applying ultrasound energy for a duration of a treatment session.

15. The method of any one of the preceding claims, wherein continuously applying ultrasound energy comprises applying ultrasound energy at a first mechanical index for a first duration, applying ultrasound energy at a second mechanical index for a second duration, and re-applying ultrasound energy at the first mechanical index without ceasing application of ultrasound energy for the duration of a treatment session.

16. The method of any one of the preceding claims, wherein the administering the nucleic acid payload or administering the plurality of microbubbles comprises administering intravenously through a peripheral vein.

17. The method of any one of the preceding claims, wherein the linear dsDNA construct is administered to the subject at a dosage between 0.4 mg/kg bodyweight to 1.5 mg/kg bodyweight.

18. The method of any one of the preceding claims, wherein the linear dsDNA construct is administered to the subject at a concentration of about 3.5 μg/μl.

19. The method of any one of the preceding claims, wherein the target cell(s) comprise hepatic cells.

20. The method of any one of the preceding claims, wherein the target cell(s) are in a liver.

21. The method of any one of claims 1-18, wherein the target cell(s) comprise renal cells.

22. The method of any one of claims 1-18, or 21, wherein the target cell(s) are in a is a kidney.

23. The method of any one of claims 1-18, wherein the target cell(s) comprise pancreatic cells.

24. The method of any one of the preceding claims, wherein the linear dsDNA construct comprises a protelomerase recognition sequence in each loop.

25. The method of claim 24, wherein the protelomerase recognition sequence comprises at least 14 nucleotides of a double stranded palindromic sequence.

26. The method of any one of the preceding claims, wherein the linear dsDNA construct comprises at least one eukaryotic promoter.

27. The method of any one of the preceding claims, wherein the linear dsDNA construct comprises at least one stem loop motif comprising a central non-complementary loop section flanked by two complementary sequences.

28. The method of any one of the preceding claims, wherein the loops are hairpin loops.

29. The method of any one of the preceding claims, wherein the microbubbles comprise a protein stabilized shell.

30. The method of any one of the preceding claims, wherein the microbubbles comprise an albumin stabilized shell.

31. The method of any one of the preceding claims, wherein the microbubbles comprise perflutren gas.

32. The method of any one of the preceding claims, wherein the microbubble is an Optison microbubble.

33. The method of any one of the preceding claims, wherein the microbubbles comprise a lipid stabilized shell.

34. The method of any one of the preceding claims, wherein a concentration of the microbubbles administered is up to about 1.2*10 microbubbles/ml.

35. The method of any one of the preceding claims, wherein the microbubbles are administered at a concentration of at least 5*10\hat{ }8 microbubbles/ml.

36. The method of any one of the preceding claims, wherein a concentration of the microbubbles administered is between 5*10\hat{ }8 and 8*10\hat{ }8 microbubbles/ml.

37. The method of the preceding claim, wherein the administering ultrasound energy occurs transcutaneously.

38. The method of any one of the preceding claims, wherein the administering the effective amount of the ultrasound energy comprises administering at least 5 ultrasound flashes at a second mechanical index which is increased relative to a first mechanical index, the ultrasound flashes at the second mechanical index being administered less than 10 seconds apart from one another.

39. The method of any one of the preceding claims, wherein the administering the effective amount of the ultrasound energy comprises administering a plurality of ultrasound flashes at an increased mechanical index, the ultrasound flashes at the increased mechanical index being administered about 1 s to about 10 s apart from one another.

40. The method of any one of the preceding claims, wherein the administering the effective amount of the ultrasound energy comprises administering a plurality of ultrasound flashes at an increased mechanical index, with each of the plurality of ultrasound flashes at the increased mechanical index being administered about 5 s to about 10 s apart from one another.

41. The method of any one of the preceding claims, wherein the administering the effective amount of the ultrasound energy comprises administering a plurality of ultrasound flashes at an increased mechanical index, the ultrasound flashes at the increased mechanical index being administered 1 s to about 5 s apart from one another.

42. The method of claims 38-41, wherein the ultrasound flash(s) is an application of ultrasound at the increased mechanical index or the second mechanical index over a time period.

43. The method of claim 42, wherein the time period is less than 1 s.

44. The method of claim 42, wherein the time period is from about 0.7 us to about 3 us (microseconds).

45. The method of claim 42, wherein the time period is about 0.72, 0.82, 0.72, 0.98, or 2.28 us.

46. The method of any one of claims 42-45, wherein the administering of the ultrasound energy of c. comprises administering at least 5 ultrasound flashes at the second mechanical index.

47. The method of any one of the preceding claims, wherein the nucleic acid payload comprises a therapeutic transgene.

48. The method of claim 47, wherein the therapeutic transgene comprises: GLP-1, INS, Reg3g, MafA, PDX-1, NUEROG3, NGN3, DRYK, DYRK1A, DYRK1B, Factor VIII, Factor IX, PKD1, PKD2, COL4A3, COL4A4, COL4A5, Klotho, Smad7, TGF-beta, SLC7A1, SLC3A9, UMOD, REN, HNF1B, MUC1, SLC12A1, KCNJ1, CLCNKA, CLCNKB, BSND, MAGED2, NPHS1, NPHS2, CTNS or combinations thereof.

49. The method of any one of claims 47-48, wherein the nucleic acid payload comprises the therapeutic transgene is coupled to a promoter sequence comprising: ApoE promoter, CAG promoter, AAT promoter, or combinations thereof.

50. The method of any one of the preceding claims, wherein the nucleic acid payload and the plurality of microbubbles are administered in a volumetric ratio of at least 1-part nucleic acid payload solution to 4 parts microbubble solution.

51. The method of any one of the preceding claims, wherein the ultrasound energy is applied at an MI of at least 0.8, 1.2, 1.4, 1.8, or 2.3.

52. A kit comprising:
a. a linear double stranded DNA (dsDNA) construct covalently closed at each of its ends by DNA loops; and
b. a plurality of microbubbles.

53. The kit of claim 52, further comprising instructions for administering to the subject, in proximity to a target cell(s) an effective amount of an ultrasound energy, sufficient to induce the formation of pores the target cell(s) by disrupting the plurality of microbubbles to result in introduction of the dsDNA into the target cell(s) and expression of the nucleic acid payload in the target cell(s).
